(19) United States
(12) Patent Application Publication
(10) Pub. No.: US 2026/0016473 A1
(43) Pub. Date: Jan. 15, 2026

(54) REAGENT FOR DETECTING SARS-RELATED CORONAVIRUS

(71) Applicant: National Institute of Advanced Industrial Science and Technology, Tokyo (JP)

(72) Inventors: Ryo NISHIHARA, Tsukuba-shi, Ibaraki (JP); Ryoji KURITA, Tsukuba-shi, Ibaraki (JP)

(21) Appl. No.: 18/994,321

(22) PCT Filed: Jul. 14, 2023
(86) PCT No.: PCT/JP2023/026063

§ 371 Date: Jan. 14, 2025

(30) Foreign Application Priority Data
Jul. 15, 2022 (JP) 2022-114168

(51) Int. Cl. G01N 33/569; C07D 487/04; G01N 33/58

(52) U.S. Cl. CPC G01N 33/56983; C07D 487/04; G01N 33/582; G01N 2333/165; G01N 2469/10

(57) ABSTRACT

The present invention relates to a compound represented by the following general formula (1), or a salt or solvate thereof:

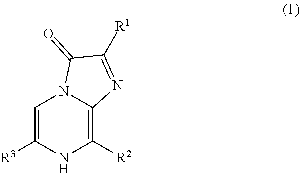

in the formula (1), R¹, R², and R³ are as defined in the description.

### TECHNICAL FIELD

The present invention relates to a novel compound or a salt or solvate thereof, a method for detecting a SARS-related coronavirus, and an agent for detecting a SARS-related coronavirus.

### BACKGROUND ART

The pandemic of a novel coronavirus infection has been caused by SARS-CoV-2 viruses that are SARS-related coronaviruses. The diagnosis of the infection requires the detection of viruses.

A current virus detection principle can be broadly classified into a polymerase chain reaction (PCR) and an antigen-antibody reaction. In the PCR, a gene of a virus is amplified, and in the antigen-antibody reaction, the specific affinity between a viral protein and immunoglobulin is utilized. In response to the spread of infection of novel coronaviruses, the miniaturization and increase in throughput of existing apparatuses merely used in molecular biology have been intensively performed to be applied to the use in clinical settings. However, the extension of an existing technique nearly reaches its limit in terms of convenience and versatility, and it is difficult to deal with an outbreak.

### CITATION LIST

### Patent Literature

PTL 1: WO2021/187531

### SUMMARY OF INVENTION

### Technical Problem

PTL 1 discloses that a specific coelenterazine analogue in which luciferin that is a substrate in a luciferin-luciferase reaction is modified can be oxidation-catalyzed by human serum albumin, causing light emission.

However, PTL 1 does not disclose a luminescent substrate capable of specifically detecting a specific virus.

An object of the present invention is to provide a novel compound or a salt or solvate thereof that can be particularly used as an agent for detecting a SARS-related coronavirus, a method for detecting a SARS-related coronavirus, and an agent for detecting a SARS-related coronavirus.

### Solution to Problem

In view of the aforementioned problems, the present inventors have intensively studied, and as a result found that a specific compound reacts with a SARS-related coronavirus to emit light, and completed the present invention.

The present invention encompasses the following aspects.
[1]A compound represented by the following general formula (1), or a salt or solvate thereof.

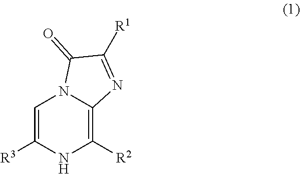

wherein R¹ is a methyl group or any one group represented by the following formula (i-1) or (i-2):

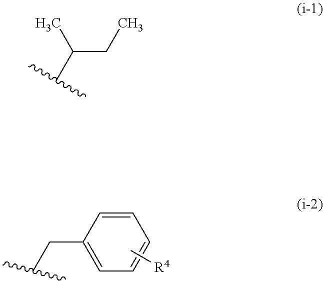

wherein R⁴ is a hydrogen atom, a hydroxy group, a fluorine atom, a methoxy group, or a trifluoromethyl group;
R² is a hydrogen atom or any one group represented by the following formula (ii):

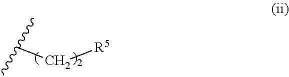

wherein n is an integer of 1 to 5, and R⁵ is any one group represented by the following formula (v-1), (v-2), or (v-3):

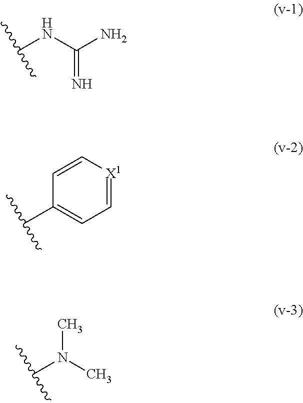

wherein X¹ is a nitrogen atom or a CH group;
R³ is any one group represented by the following formula (iii-1) or (iii-2):

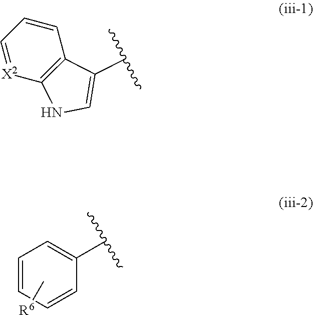

wherein X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5):

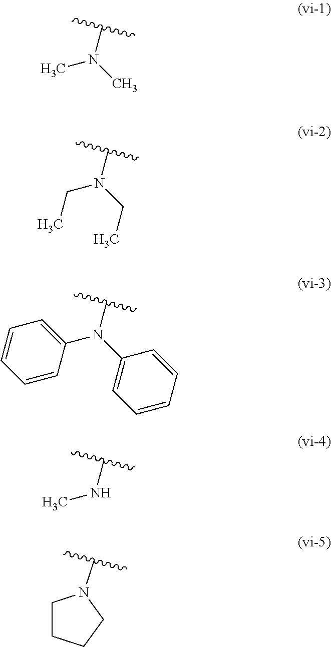

the compound satisfying the following condition (A-i), (A-ii), or (A-iii):
(A-i) the R¹ is a group represented by the following formula (i-2):

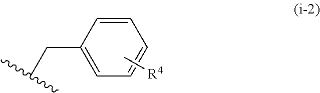

wherein R⁴ is the same as described above; and the R² is a group represented by the following formula (ii):

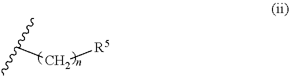

wherein n and R⁵ are the same as described above;
(A-ii) the R¹ is a group represented by the following formula (i-1):

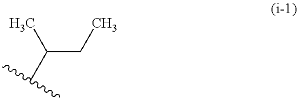 [SMILES: *C(C)CC]

provided that native Cypridina luciferin is excluded; and
(A-iii) the R¹ is a methyl group and the R² is a group represented by the following formula (ii):

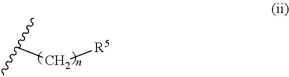

wherein n and R⁵ are the same as described above.
[2] The compound or the salt or solvate thereof according to [1], wherein the compound represented by the general formula (1) is a compound represented by any one of the following formulae.

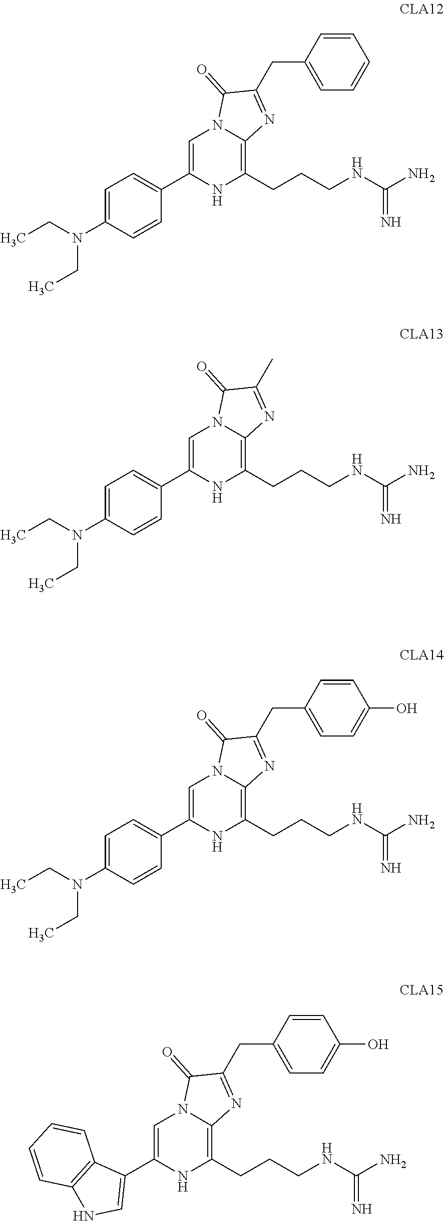

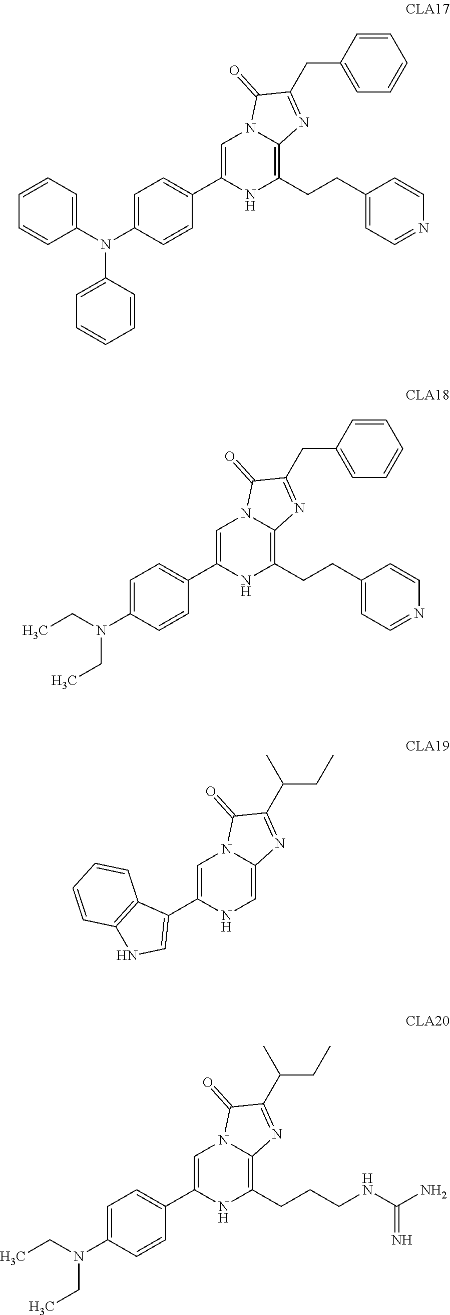

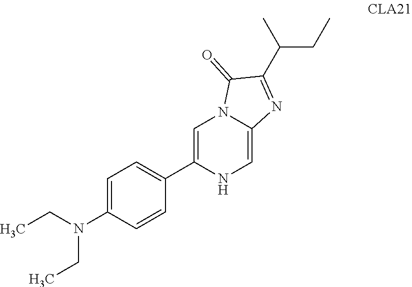

[3]A method for detecting a SARS-related coronavirus including a step of bringing a compound represented by the following general formula (2) or a salt or solvate thereof into contact with a biological sample collected from a subject:

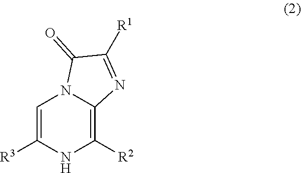

wherein R¹ is a methyl group or any one group represented by the following formula (i-1) or (i-2):

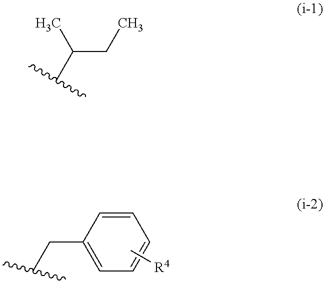

wherein R⁴ is a hydrogen atom, a hydroxy group, a fluorine atom, a methoxy group, or a trifluoromethyl group; R² is a hydrogen atom or any one group represented by the following formula (ii):

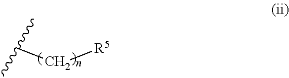

wherein n is an integer of 1 to 5, and R⁵ is any one group represented by the following formula (v-1), (v-2), or (v-3):

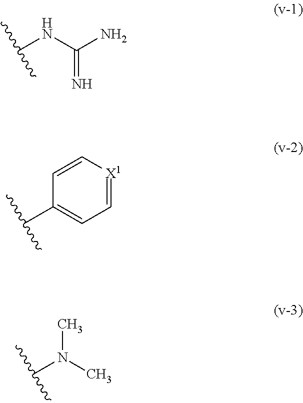

wherein X¹ is a nitrogen atom or a CH group; and
R³ is any one group represented by the following formula (iii-1) or (iii-2):

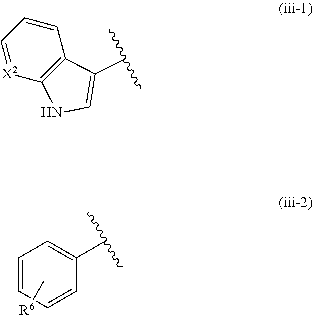

wherein X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5).

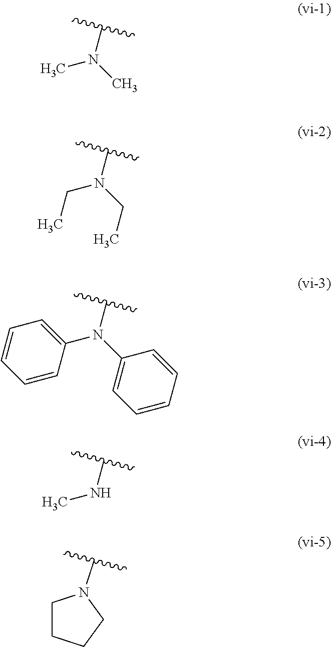

[4] The method for detecting a SARS-related coronavirus according to [3], wherein the compound represented by the general formula (2) is a compound represented by any of the following formulae.

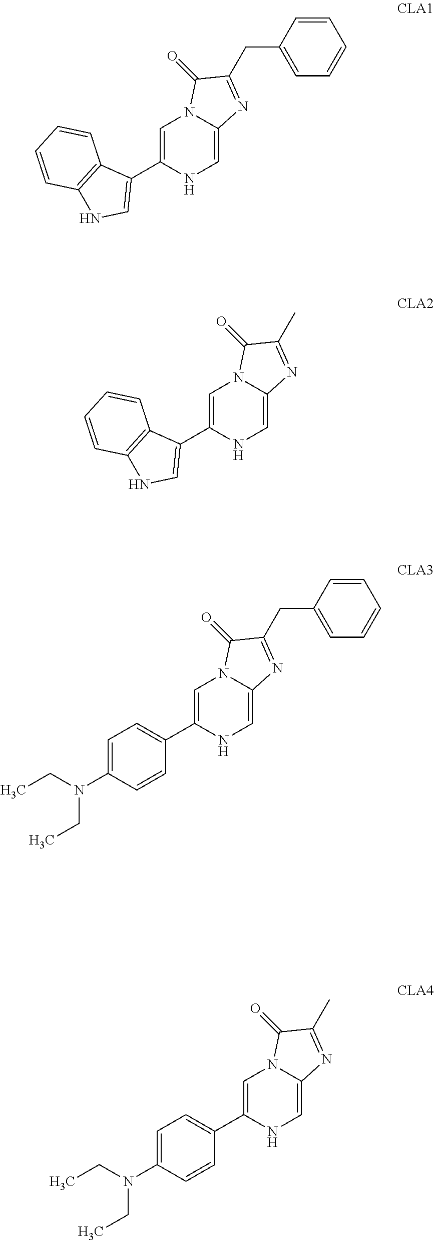

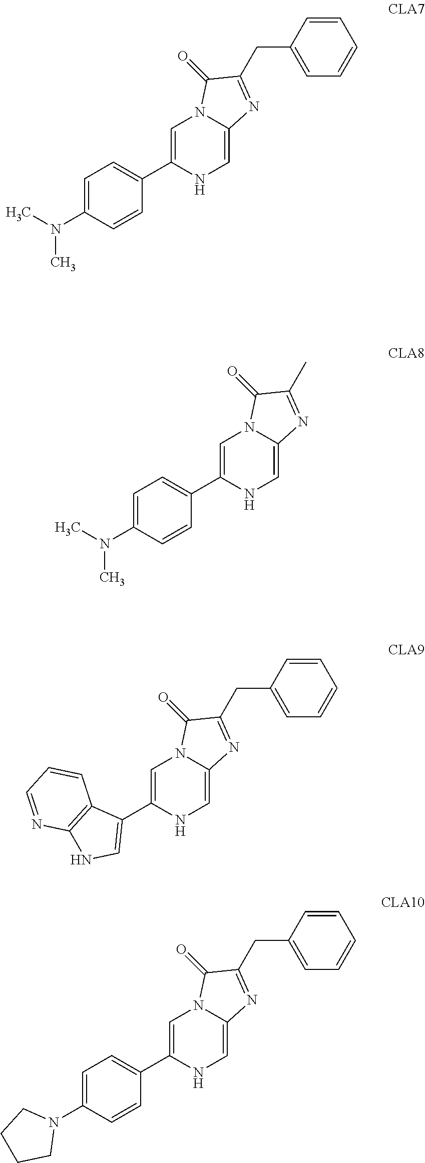

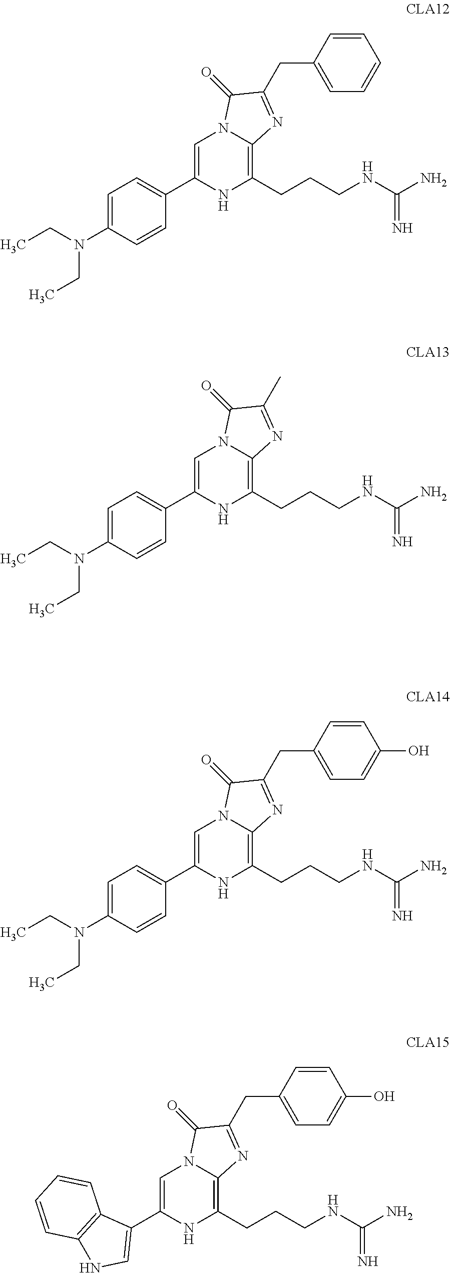

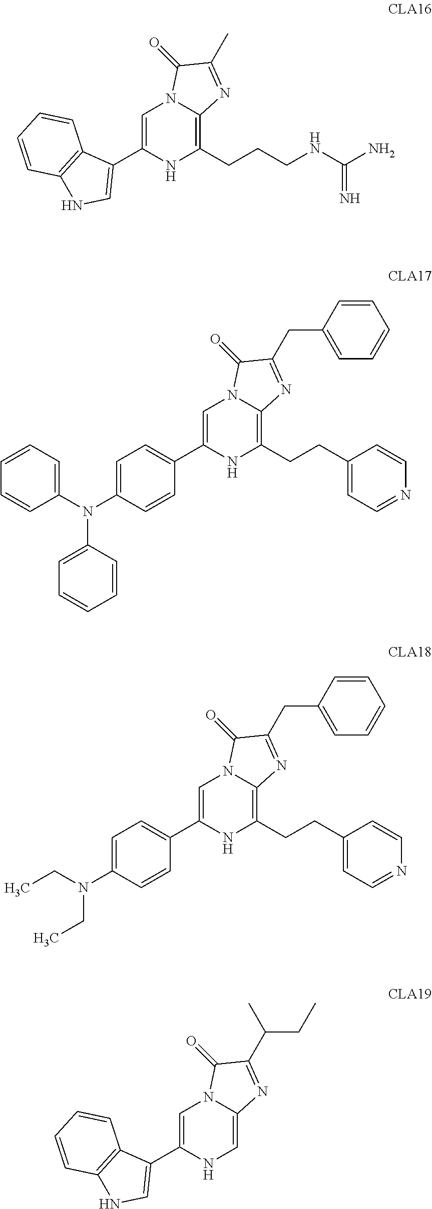

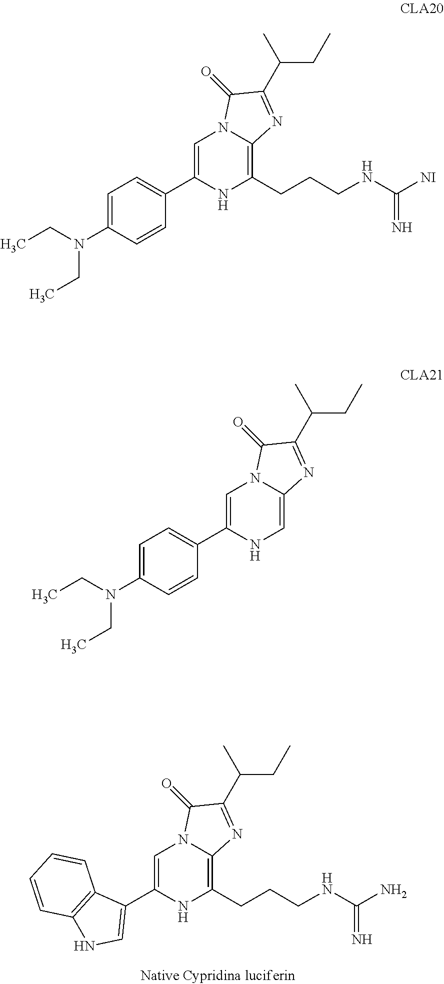

[5] The method for detecting a SARS-related coronavirus according to [3], wherein the compound represented by the general formula (2) satisfies at least two of the following conditions (B-i), (B-ii), and (B-iii):
(B-i) the R¹ is a group represented by the following formula (i-1):

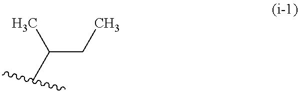 [SMILES: *C(C)CC]

(B-ii) the R² is a group represented by the following formula (ii):

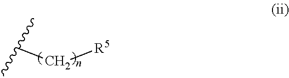

wherein n and R⁵ are the same as described above; and
(B-iii) the R³ is a group represented by the following formula (iii-2-2).

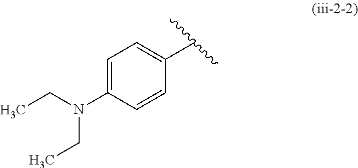 [SMILES: *c1ccc(N(CC)CC)cc1]

[6] The method for detecting a SARS-related coronavirus according to any one of [3] to [5], further including a step of measuring luminescence after the contact step.
[7] An agent for detecting a SARS-related coronavirus including a compound represented by the following general formula (2) or a salt or solvate thereof:

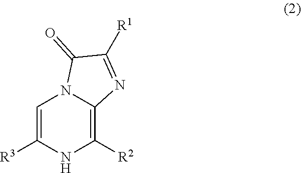

wherein R¹ is a methyl group or any one of groups represented by the following formulae:

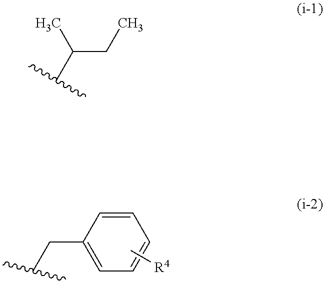

wherein R⁴ is a hydrogen atom, a hydroxy group, a fluorine atom, a methoxy group, or a trifluoromethyl group;
R² is a hydrogen atom or any one group represented by the following formula:

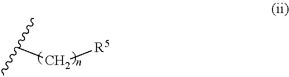

wherein n is an integer of 1 to 5, and R⁵ is any one of groups represented by the following formulae:

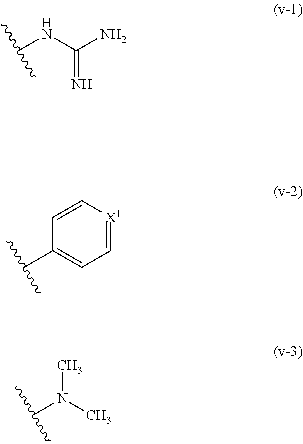

wherein X¹ is a nitrogen atom or a CH group; and
R³ is any one of groups represented by the following formulae:

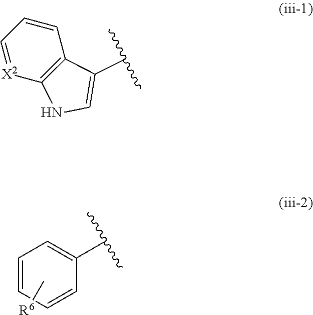

wherein X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5).

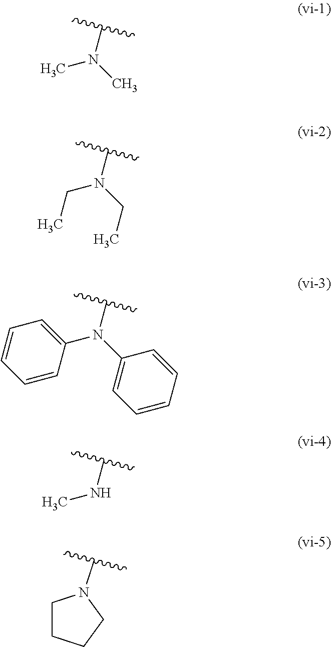

### Advantageous Effects of Invention

According to the present invention, a novel compound or a salt or solvate thereof, a method for detecting a SARS-related coronavirus, and an agent for detecting a SARS-related coronavirus are provided.

### BRIEF DESCRIPTION OF DRAWINGS

FIG. 1(i) represents results of measurement of luminescence activities of domains of a spike protein of a SARS-CoV-2 virus for native Cypridina luciferin and FIG. 1(ii) represents results of measurement of luminescence activities of spike proteins of other viruses for native Cypridina luciferin.

FIG. 2(i) represents cross-reactivity between native Cypridina luciferin and immunoglobulin protein IgA. FIG. 2(ii) represents correlations between the concentration of a spike protein of a SARS-CoV-2 virus and the luminescence intensity of native Cypridina luciferin.

FIG. 3 represents cross-reactivity between native Cypridina luciferin and various proteins.

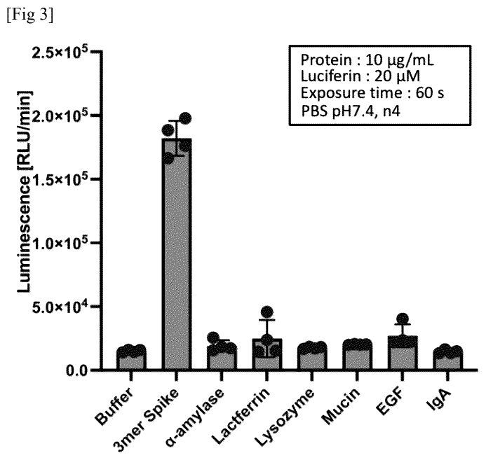

### DESCRIPTION OF EMBODIMENTS

### 1. Compound or Salt or Solvate Thereof

A compound of the present invention is represented by the following general formula (1). The compound of the present invention has an imidazopyrazinone ring as a main backbone and includes a derivative of native Cypridina luciferin. In this description, a broken line portion in a chemical formula represents a bond position.

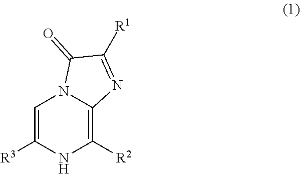

In the formula, R¹ is a methyl group or any one group represented by the following formula (i-1) or (i-2):

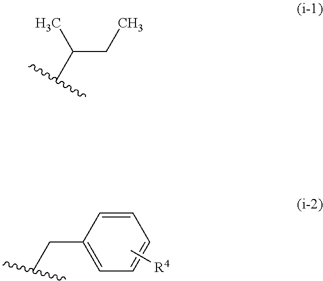

in which R⁴ is a hydrogen atom, a hydroxy group, a fluorine atom, a methoxy group, or a trifluoromethyl group;
R² is a hydrogen atom or any one group represented by the following formula (ii):

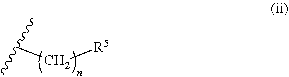

in which n is an integer of 1 to 5, and R⁵ is any one group represented by the following formula (v-1), (v-2), or (v-3):

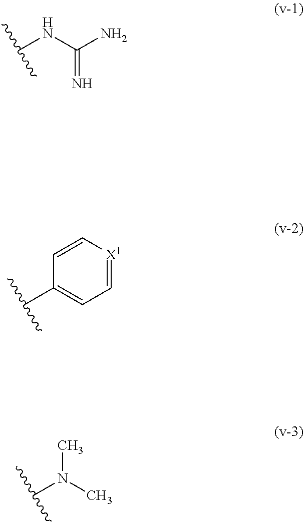

in which X¹ is a nitrogen atom or a CH group;
R³ is any one group represented by the following formula (iii-1) or (iii-2):

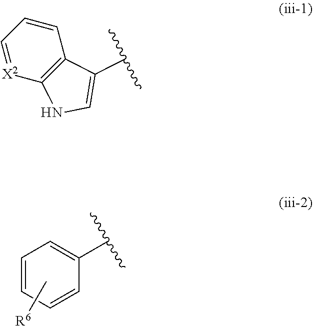

in which X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5).

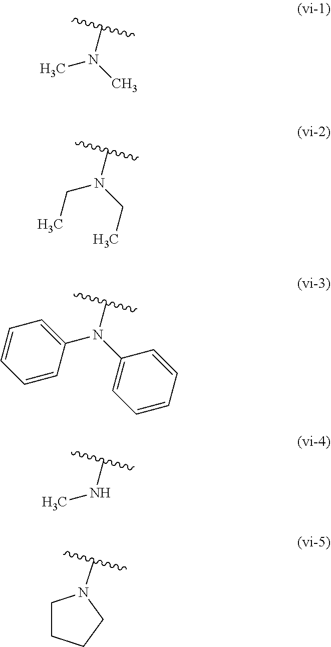

In addition, the following condition (A-i), (A-ii), or (A-iii) is satisfied.
(A-i) The R¹ is a group represented by the following formula (i-2):

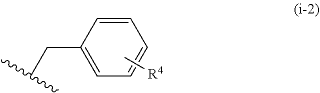

in which R⁴ is the same as described above; and the R² is a group represented by the following formula (ii):

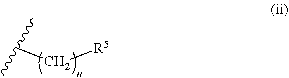

in which n and R⁵ are the same as described above.
(A-ii) The R¹ is a group represented by the following formula (i-1), provided that native Cypridina luciferin is excluded.

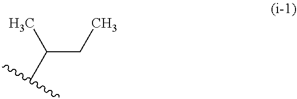 [SMILES: *C(C)CC]

(A-iii) The R¹ is a methyl group and the R² is a group represented by the following formula (ii):

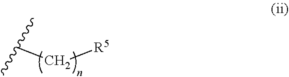

in which n and R⁵ are the same as described above.

The aforementioned formula (i-1) has an asymmetric carbon C*¹ shown below.

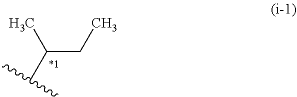 [SMILES: *C(C)CC]

The configuration of C*¹ may be R configuration represented by the following formula (i-1-R) or the following formula (i-1-S). Moreover, the configuration of C*¹ may be one configuration alone or a mixture of the configurations. Examples of a mixture of an R configuration and an S configuration include a racemate including an equal amount of each configuration.

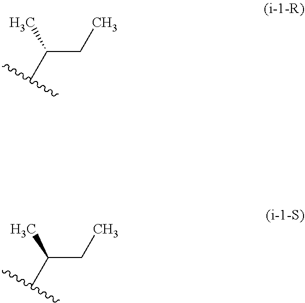

In the aforementioned formula (i-2), when R⁴ is a hydroxy group, a fluorine atom, a methoxy group, or a trifluoromethyl group, a bond position of R⁴ in the benzene ring skeleton of the formula (i-2) may be any of an ortho position, a meta position, and a para position, and preferably a para position, with respect to the methylene group on another side.

R⁴ is preferably a hydrogen atom or a hydroxy group.

A preferable aspect of the group represented by the formula (i-2) includes groups represented by the following formulae (i-2-1) and (i-2-2).

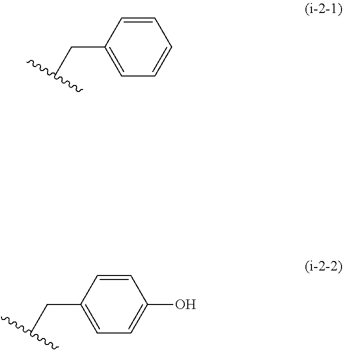 [SMILES: *Cc1ccccc1 ; *Cc1ccc(O)cc1]

The n is an integer of 1 to 5, and preferably 2 or 3.

The group represented by the aforementioned formula (v-2) is a group represented by the following formula (v-2-1) or (v-2-2).

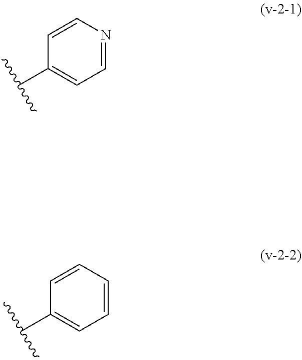 [SMILES: *c1ccncc1 ; *c1ccccc1]

A preferable aspect of the R² includes a hydrogen atom or a group represented by the following formula (ii-1) or (ii-2).

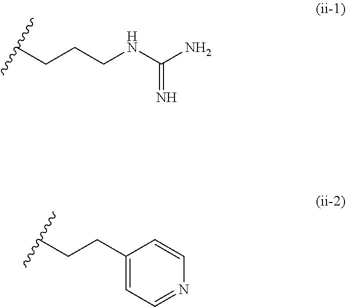 [SMILES: *CCCNC(=N)N ; *CCc1ccncc1]

The group represented by the aforementioned formula (iii-1) is a group represented by the following formula (iii-1-1) or (iii-1-2).

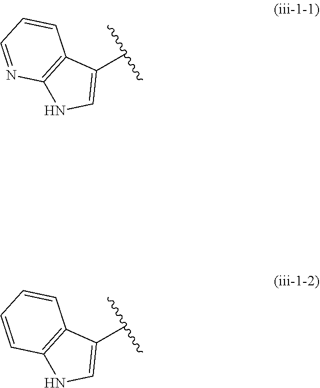 [SMILES: *c1c[nH]c2ncccc12 ; *c1c[nH]c2ccccc12]

In the aforementioned formula (iii-2), a bond position of R⁶ in the benzene ring skeleton may be any of an ortho position, a meta position, and a para position, and preferably a para position, with respect to the imidazopyrazinone ring on another side.

A preferable aspect of the R³ includes a group represented by the aforementioned formula (iii-1-1) or (iii-1-2) or the following formula (iii-2-1), (iii-2-2), (iii-2-3), or (iii-2-4).

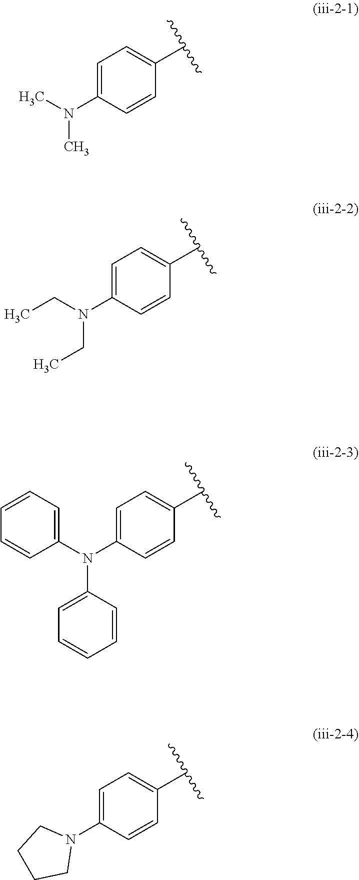

Native Cypridina luciferin is a compound represented by the following formula, and is not included in the compound satisfying the condition (A-ii).

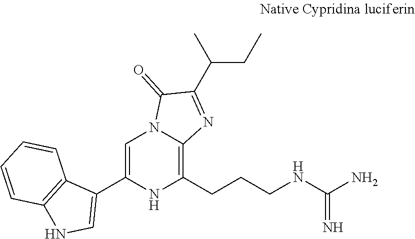 [SMILES: CCC(C)c1nc2c(CCCNC(=N)N)[nH]c(-c3c[nH]c4ccccc34)cn-2c1=O]

A preferable aspect of the compound represented by the aforementioned general formula (1) includes a compound represented by any of the following formulae.

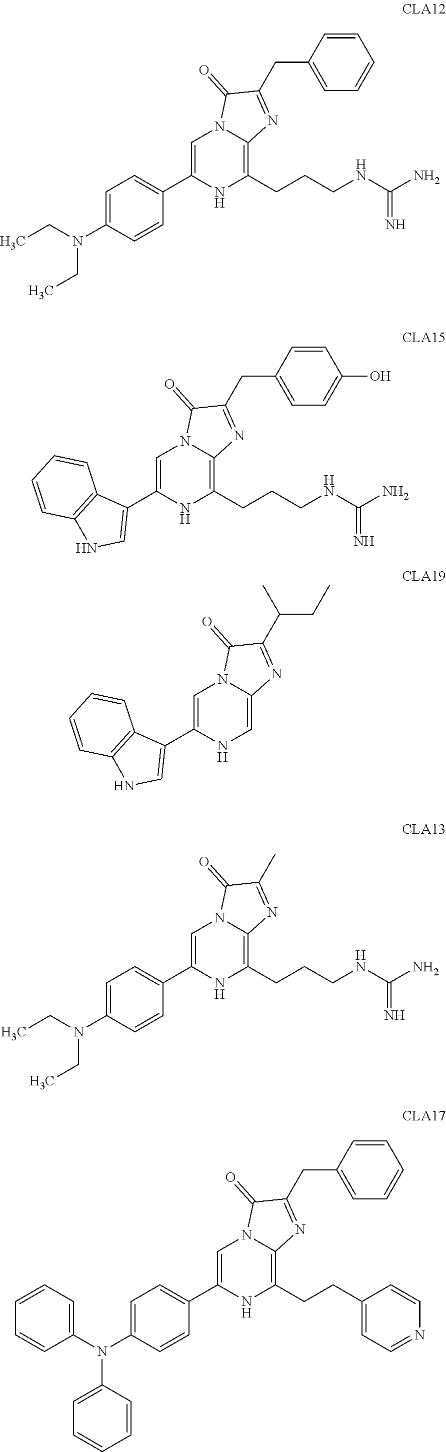

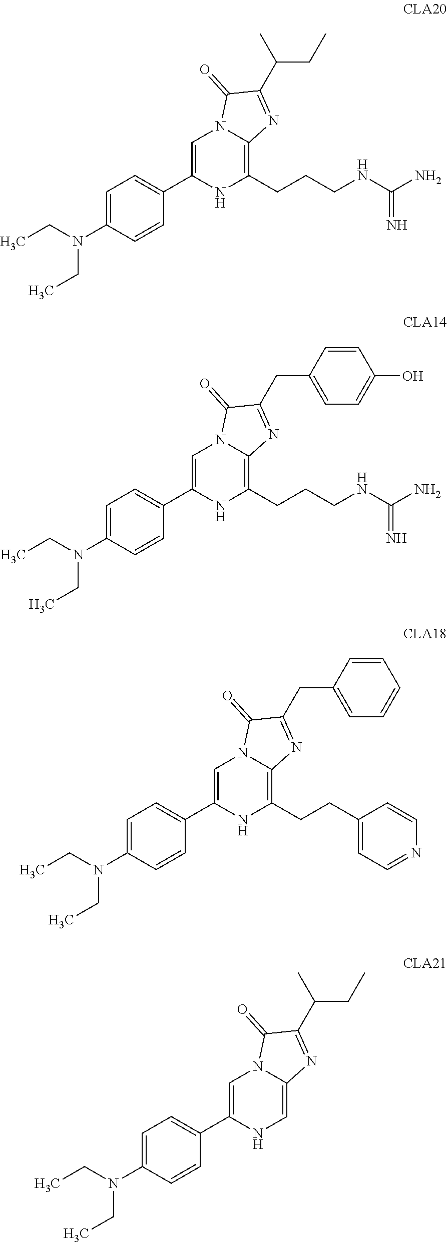

The present invention encompasses a salt of the compound represented by the general formula (1). Such a salt is not particularly limited. Specific examples thereof include acid addition salts such as a hydrohalic acid salt (e.g., hydrochloride, hydrobromide, and hydroiodide), an inorganic acid salt (e.g., sulfate, nitrate, perchlorate, phosphate, carbonate, and bicarbonate), an organic carboxylic acid salt (e.g., acetate, trifluoroacetate, maleate, tartarate, fumarate, and citrate), and an organic sulfonic acid salt (e.g., methanesulfonate, trifluoromethanesulfonate, ethanesulfonate, benzenesulfonate, toluenesulfonate, and camphorsulfonate); and base addition salts such as a quaternary amine salt, an alkali metal salt (e.g., a sodium salt and a potassium salt), and an alkaline earth metal salt (e.g., a magnesium salt, and a calcium salt).

The present invention encompasses a hydrate and solvate of the compound represented by the general formula (1). Examples of the solvate include solvates with ethanol.

### (Production Method)

The compound of the present invention can be produced, for example, by condensation of a coelenteramine derivative (x1) and a ketoacetal compound (x2-1) or a diacetyl compound (x2-2) as shown in Scheme 1 or 2.

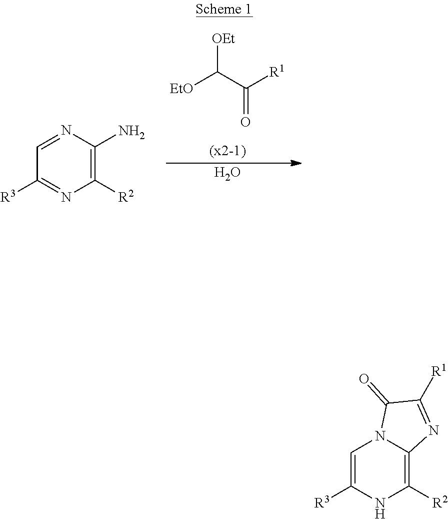

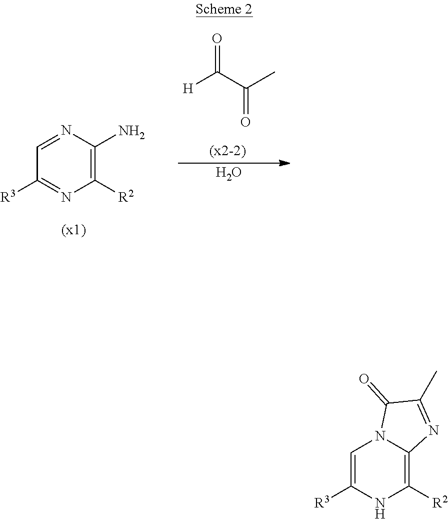

In the formulae, R¹, R², and R³ are the same as described above.

In the reaction, the ketoacetal compound (x2-1) or methylglyoxal (x2-2) is used in an amount of about 1 to 4 mol with respect to 1 mol of the coelenteramine derivative (x1) in the presence of water. When the reaction is performed in the presence of an acid at a temperature of about 65 to 100° C. for 1 to 5 hours, the reaction advantageously proceeds.

The reaction more advantageously proceeds in a solvent. Examples of the solvent include ethanol.

The compound of the present invention or the salt or solvate thereof is useful, for example, as an agent for detecting a SARS-related coronavirus, and can be suitably used as a luminescent substrate in a method for detecting a SARS-related coronavirus described below.

Note that the compound of the present invention may not function as a luminescent substrate in a luciferin-luciferase reaction although the compound is a derivative of Cypridina luciferin.

### 2. Method for Detecting SARS-Related Coronavirus

A method for detecting a SARS-related coronavirus of the present invention includes a step of bringing a compound represented by the following general formula (2) or a salt or solvate thereof into contact with a biological sample collected from a subject.

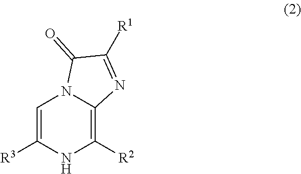

In the formula, R¹ is a methyl group or any one group represented by the following formula (i-1) or (i-2):

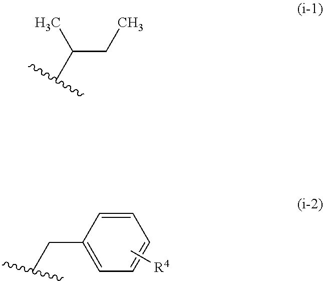

in which R⁴ is a hydrogen atom, a hydroxy group, a fluorine atom, a methoxy group, or a trifluoromethyl group;
R² is a hydrogen atom or any one group represented by the following formula (ii):

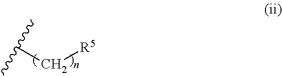

in which n is an integer of 1 to 5, and R⁵ is any one group represented by the following formula (v-1), (v-2), or (v-3):

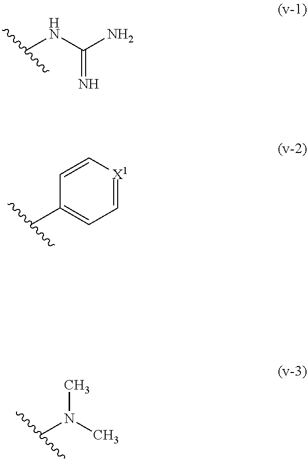

in which X¹ is a nitrogen atom or a CH group; and
R³ is any one group represented by the following formula (iii-1) or (iii-2):

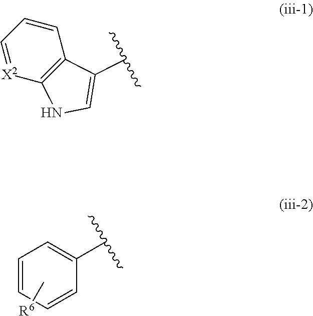

in which X² is a nitrogen atom or a CH group, R⁶ is a hydroxy group, a methoxy group, a methyl group, a trifluoromethyl group, or any one group represented by the following formula (vi-1), (vi-2), (vi-3), (vi-4), or (vi-5).

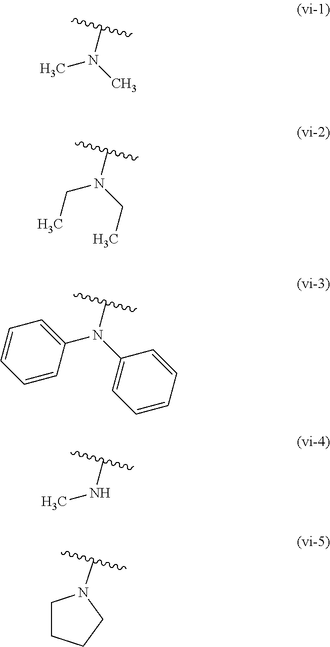

### (Compound and the Like)

In the method for detecting a SARS-related coronavirus of the present invention, the compound represented by the aforementioned general formula (2), or the salt or solvate thereof is used as a luminescent substrate.

In the compound represented by the general formula (2), R¹, R², and R³ are the same as those in the compound represented by the aforementioned general formula (1), and preferable aspects thereof are also the same.

The compound represented by the general formula (2) can be produced in the same manner for the compound represented by the general formula (1).

A preferable aspect of the compound represented by the general formula (2) includes a compound represented by any of the following formulae.

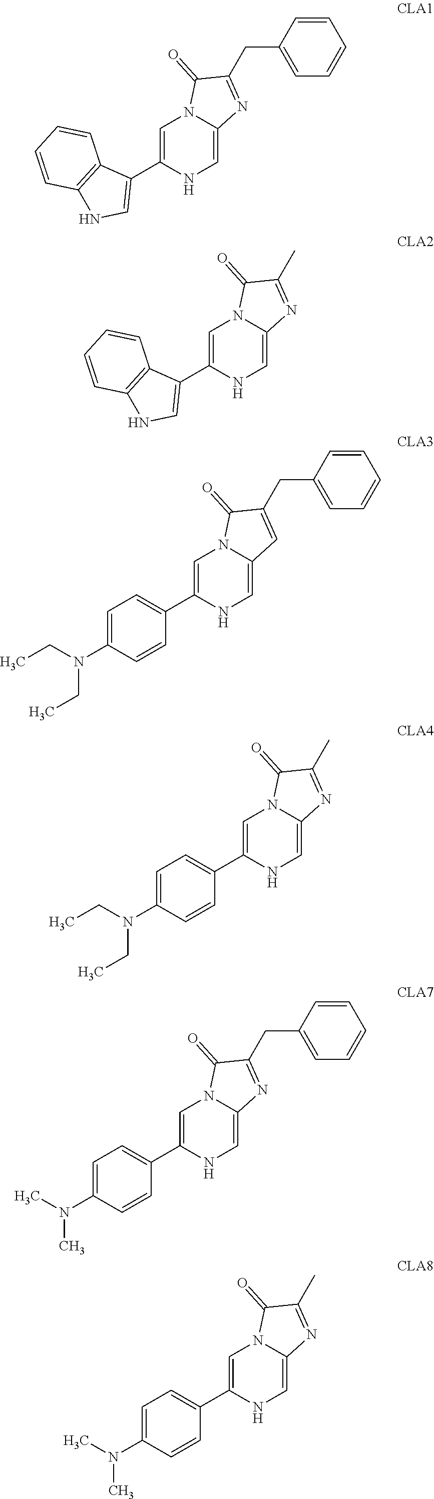

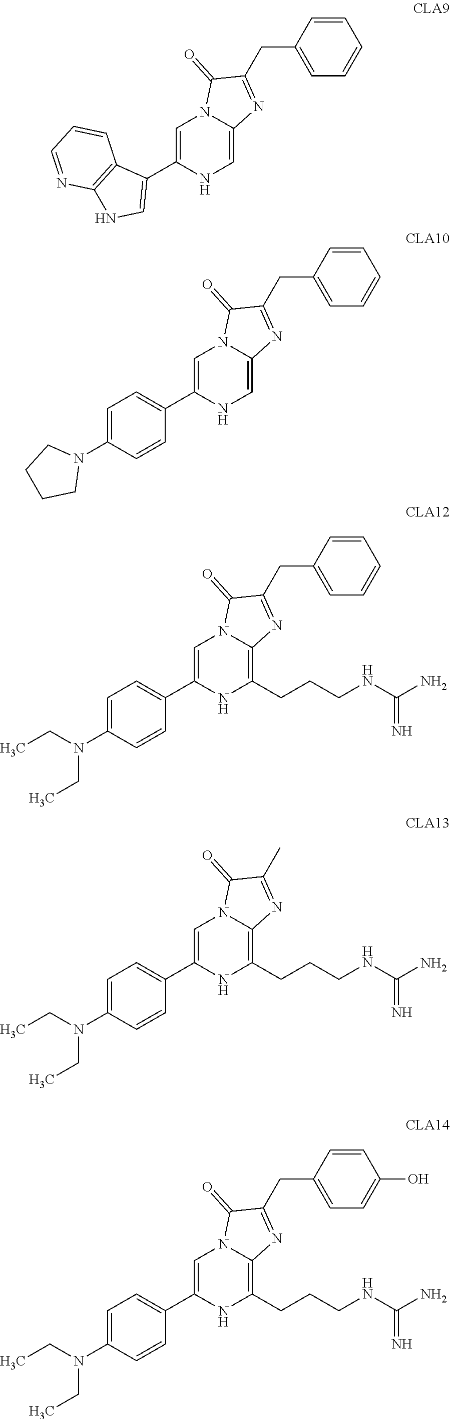

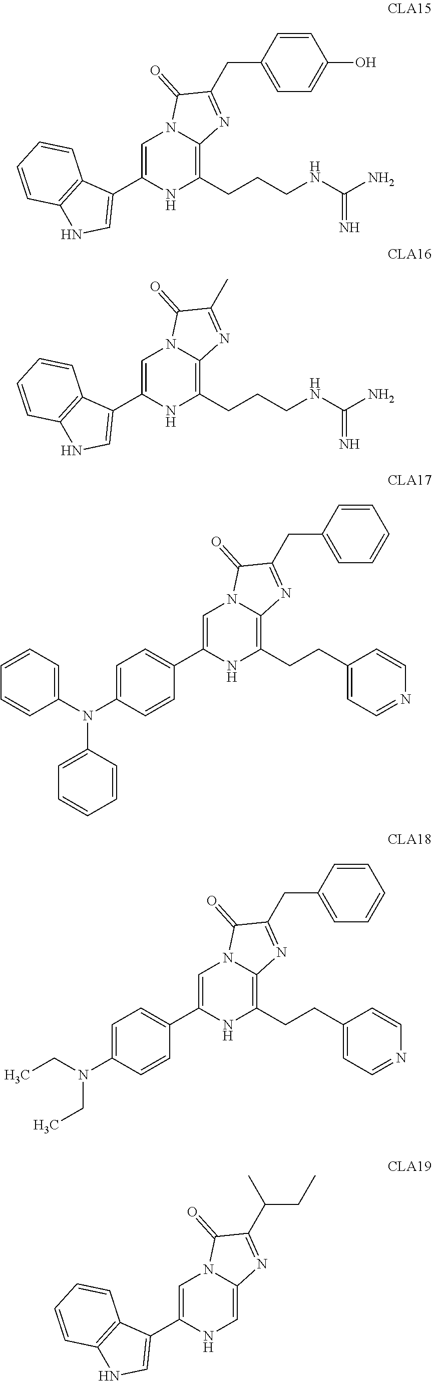

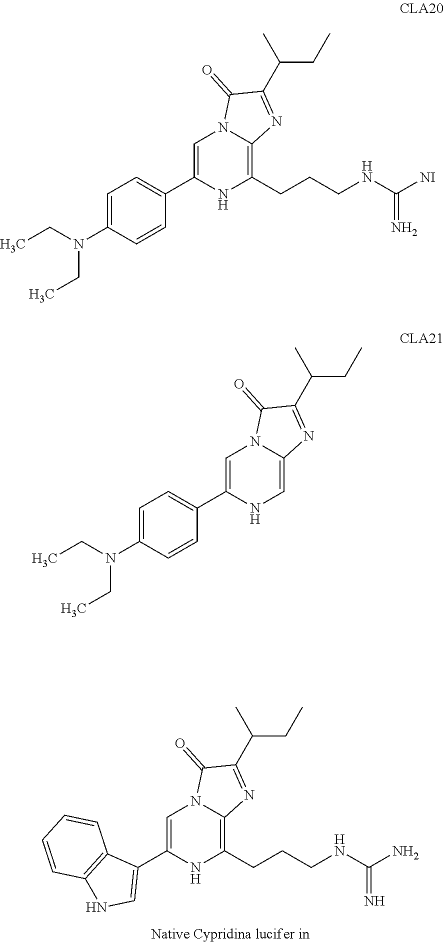

In a more preferable aspect of the method for detecting a SARS-related coronavirus of the present invention, the compound represented by the general formula (2) satisfies at least two of the following conditions (B-i), (B-ii), and (B-iii):
(B-i) The R¹ is a group represented by the following formula (i-1):

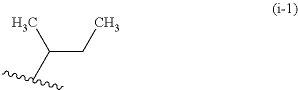 [SMILES: *C(C)CC]

(B-ii) The R² is a group represented by the following formula (ii):

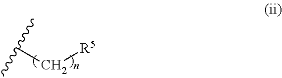

in which n and R⁵ are the same as described above; and
(B-iii) The R³ is a group represented by the following formula (iii-2-2).

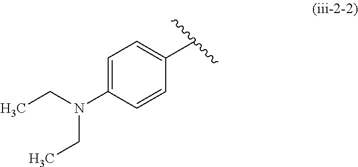 [SMILES: *c1ccc(N(CC)CC)cc1]

Examples of the compound satisfying at least two of the conditions (B-i), (B-ii), and (B-iii) include a compound represented by any of the following formulae.

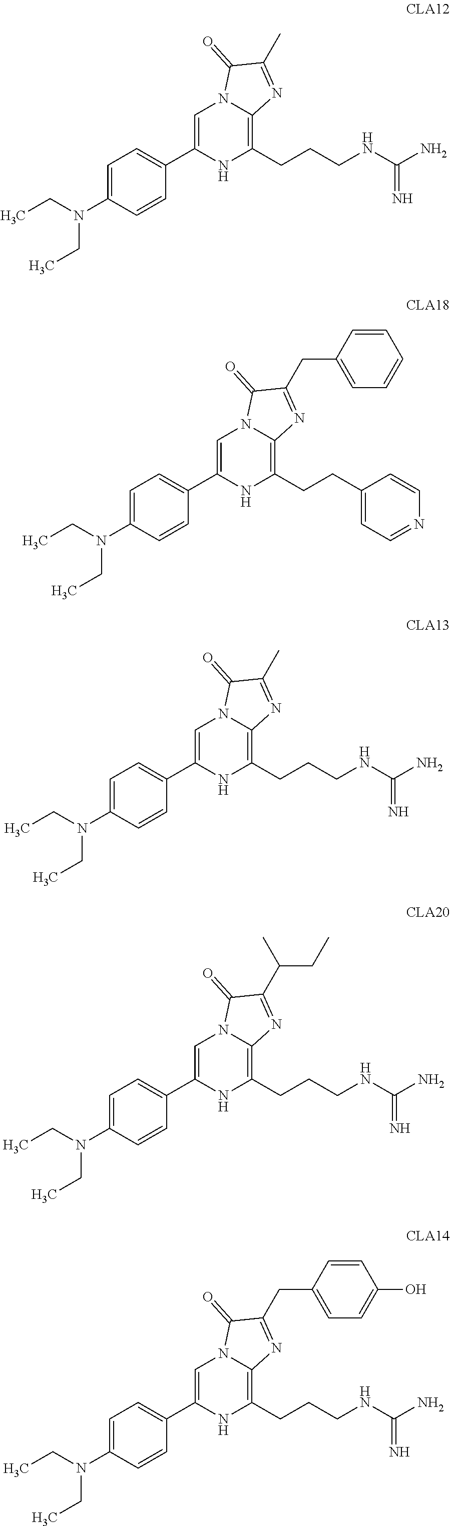 [SMILES: CCN(CC)c1ccc(-c2cn3c(=O)c(C)nc-3c(CCCNC(=N)N)[nH]2)cc1 ; CCN(CC)c1ccc(-c2cn3c(=O)c(Cc4ccccc4)nc-3c(CCc3ccncc3)[nH]2)cc1 ; CCN(CC)c1ccc(-c2cn3c(=O)c(C)nc-3c(CCCNC(=N)N)[nH]2)cc1 ; CCC(C)c1nc2c(CCCNC(=N)N)[nH]c(-c3ccc(N(CC)CC)cc3)cn-2c1=O ; CCN(CC)c1ccc(-c2cn3c(=O)c(Cc4ccc(O)cc4)nc-3c(CCCNC(=N)N)[nH]2)cc1]

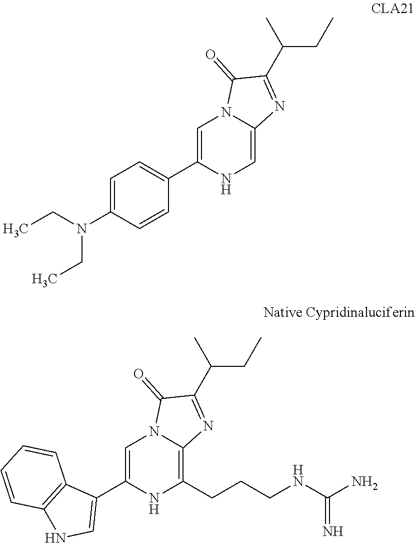 [SMILES: CCC(C)c1nc2c[nH]c(-c3ccc(N(CC)CC)cc3)cn-2c1=O ; CCC(C)c1nc2c(CCCNC(=N)N)[nH]c(-c3c[nH]c4ccccc34)cn-2c1=O]

### (SARS-Related Coronavirus)

A SARS-related coronavirus belongs to Betacoronavirus and includes SARS coronavirus-2 (Severe acute respiratory syndrome coronavirus 2; SARS-CoV-2) and SARS coronavirus (SARS-CoV).

SARS-CoV-2 is considered to cause an acute respiratory disease (COVID-19) and has caused the pandemic of COVID-19.

A SARS-related coronavirus is discriminated from MERS coronavirus, which causes middle east respiratory syndrome (MERS).

### (Subject)

A subject is not particularly limited as long as it is a subject to be infected with a SARS-related coronavirus. Specifically, the subject includes: human; primates other than human, including non-human primate such as chimpanzee, other anthropoid, and species of monkey; domestic animals such as cattle, sheep, pig, goat, and horse; domestic mammals such as dog and cat; and small animals or experimental animals including rodents such as mouse, rat, and guinea pig, and is preferably human. In addition, the subject includes an adult, a young children, and a neonate.

A preferable aspect of the subject is a subject suspected to be infected with a SARS-related coronavirus.

### (Biological Sample)

As a sample, a biological sample derived from the subject can be used as long as it is a sample in which a SARS-related coronavirus may be present. Specific examples thereof include cells and body fluid samples derived from the subject.

Examples of cells and biological tissues include oral mucosa cells, nasal cavity mucosa cells, and epidermis, which are easily sampled.

Examples of the body fluid samples include samples derived from blood, lymph, urine, sweat, saliva, nasal discharge, and tear.

### (Contact Step)

The method for detecting a SARS-related coronavirus of the present invention includes a step of bringing the compound represented by the aforementioned general formula (2) or the salt or solvate thereof into contact with the biological sample collected from the subject.

As a contact condition, reaction conditions such as the pH and salt concentration of a reaction solution, and a reaction temperature can be set according to a luciferin-luciferase reaction.

The concentration of the compound represented by the general formula (2) or the salt or solvate thereof, which is used, is not particularly limited, and is preferably 5 to 100 M, and more preferably 20 to 50 μM. A concentration that is equal to or more the aforementioned lower limit is preferred since a sufficient luminescent reaction rate is considered to be achieved. A concentration that is equal to or less than the aforementioned upper limit is preferred from the viewpoint of solubility of the compound and the like.

The reaction time is not particularly limited, and may be, for example, 10 seconds to 10 minutes, more preferably 30 seconds to 5 minutes, and further preferably 1 minute to 3 minutes since the luminescent reaction rapidly proceeds.

It is considered that the aforementioned compound reacts with a spike protein of a SARS-related coronavirus, the oxidized form of the compound transits to an excited state, and light is emitted during transition from the excited state to the ground state.

Accordingly, when a SARS-related coronavirus is present in the biological sample, luminescence is observed after contact. That is, when luminescence is measured, the subject can be determined to be infected with a SARS-related coronavirus.

It is preferable that the method for detecting a SARS-related coronavirus of the present invention further include a step of measuring luminescence after the aforementioned contact step.

The step of measuring luminescence can be performed using a general luminescence measurement apparatus. Examples of the luminescence measurement apparatus include a luminometer, a microscope equipped with a luminescence detection means, and a luminescence photographing apparatus.

Thus, a SARS-related coronavirus is detected.

For the subject determined to be infected with a SARS-related coronavirus, a necessary measure such as an infection prevention measure against other subjects and a treatment may be conducted.

### 3. Agent for Detecting SARS-Related Coronavirus

The present invention also provides a detecting agent containing the compound represented by the general formula (2) or the salt or solvate thereof.

The detecting agent is provided, for example, in the form of composition. The composition may contain another component as required. Examples of the other component include a base, a carrier, a solvent, a dispersant, an emulsifier, a buffer, a stabilizer, an excipient, a binder, a disintegrant, a lubricant, a thickener, a moisturizer, a colorant, a perfume, and a chelating agent.

Moreover, the detecting agent is provided, for example, in the form of a kit for detecting a SARS-related coronavirus. The kit may contain various reagents (e.g., a reaction solution), an appliance (e.g., an appliance for collecting and storing a biological sample), and the like. Furthermore, a manual in which the method for detecting a SARS-related coronavirus of the present invention is described may be included as a method for using the kit.

### EXAMPLES

The present invention will be described more specifically with reference to Examples, but the present invention is not limited to these examples.

### 1. Synthesis Example

Reagents were each purchased from FUJIFILM Wako Pure Chemical Corporation, Kanto Chemical Co., Inc., Tokyo Chemical Industry Co., Ltd., BLD Pharmatech Ltd., or Sigma-Aldrich Co. LLC., and used directly without purification.

In ¹H-NMR and ¹³C-NMR, Avance III-500 manufactured by Bruker Corporation was used as a measurement apparatus. Tetramethylsilane (TMS, 0 ppm) was used as an internal standard. A coupling constant (J) was expressed in Hz. Abbreviations s, d, t, q, m, and br refer to singlet, doublet, triplet, quartet, multiplet, and broad, respectively.

### Outline

The compound of the present invention was synthesized by condensation of a coelenteramine derivative and a ketoacetal compound or diacetyl as shown in Scheme 1 or 2.

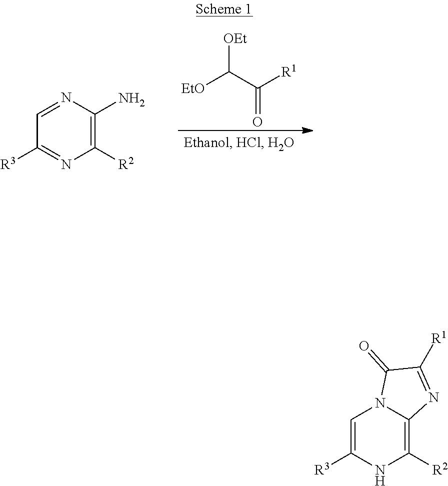

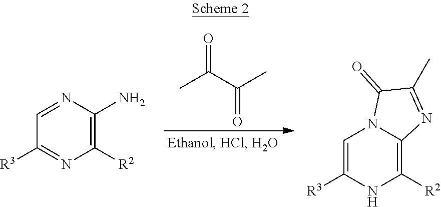

In the formulae, R¹, R², and R³ are the same as described above.

The coelenteramine derivative was synthesized by the following Schemes 3 to 5.

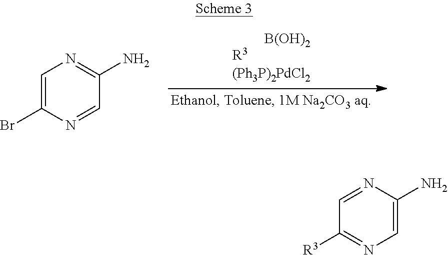

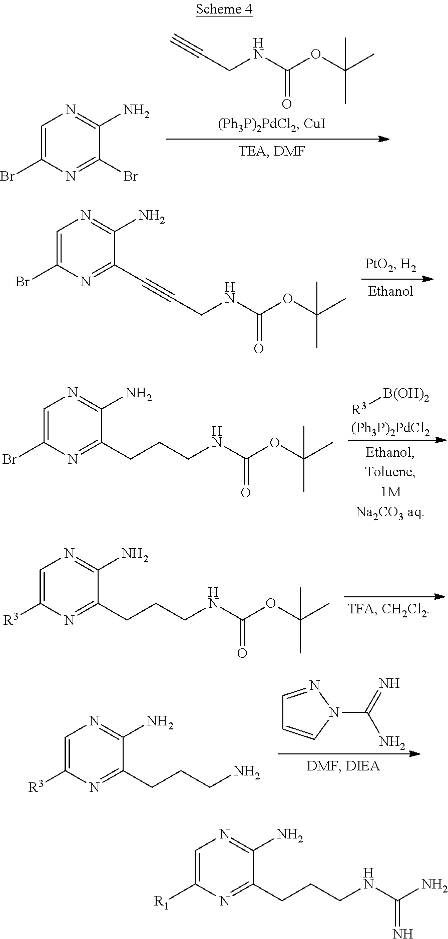

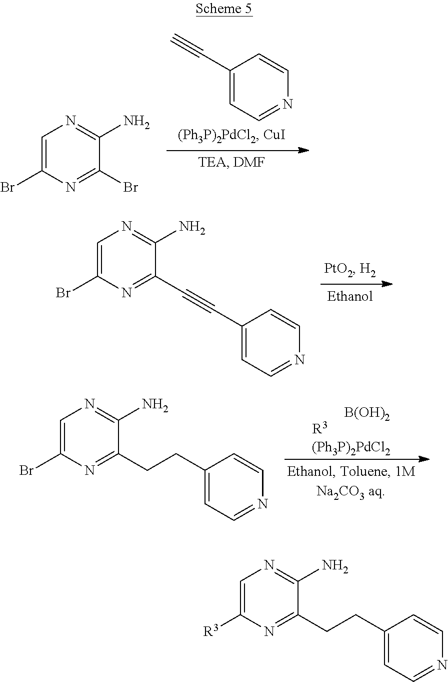

In the formulae, R¹, R², and R³ are the same as described above.

### Synthesis Example 1: Synthesis of 2-benzyl-6-(1H-indol-3-yl)imidazo[1,2-a]pyrazin-3(7H)-one (CLA1)

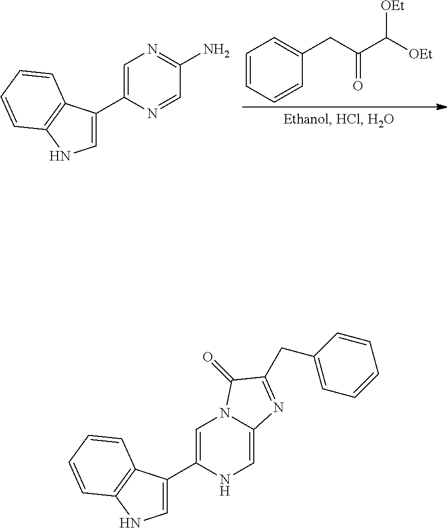

Under an argon atmosphere, 5-(1H-indol-3-yl)pyrazin-2-amine (40.0 mg, 0.19 mmol, 1 eq.) and 1,1-diethoxy-3-phenylpropan-2-one (50.7 mg, 0.22 mmol, 1.2 eq.) were dissolved in ethanol (3 mL) and ultrapure water (Milli-Q) (0.3 mL) and then cooled to 0° C. The reaction solution was degassed in a vacuum, concentrated hydrochloric acid (0.1 mL) was added, and the mixture was stirred at 80° C. overnight.

The mixture was cooled to room temperature and then concentrated under reduced pressure, and the residue was purified by column chromatography (eluent: methylene chloride/methanol=20/1) to obtain 2-benzyl-6-(1H-indol-3-yl)imidazo[1,2-a]pyrazin-3(7H)-one (CLA1) (49.8 mg, 77%).

¹H-NMR (500 MHz, CD₃OD): δ (ppm)=7.78 (t, J=11.8 Hz, 2H), 7.66 (s, 1H), 7.56 (s, 1H), 7.34 (d, J=8.0 Hz, 1H), 7.23 (d, J=7.2 Hz, 2H), 7.18-7.05 (m, 5H), 4.04 (s, 2H).

¹³C-NMR (125 MHz, CD₃OD): δ (ppm)=35.43, 108.70, 113.93, 120.80, 122.59, 124.64, 126.42, 128.32, 130.39, 130.83, 139.33, 140.45.

### Synthesis Example 2: Synthesis of 2-(sec-butyl)-6-(1H-indol-3-yl)imidazo[1,2-a]pyrazin-3(7H)-one (CLA19)

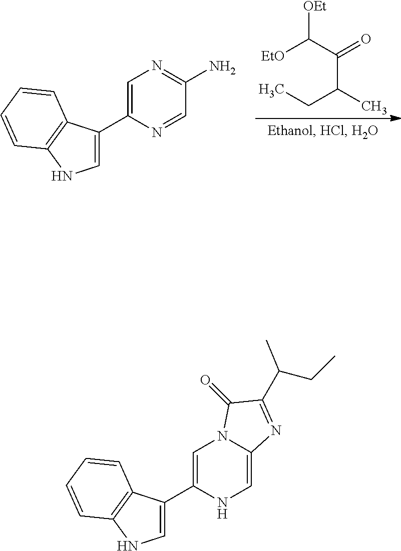 [SMILES: Nc1cnc(-c2c[nH]c3ccccc23)cn1 ; CCOC(OCC)C(=O)C(C)CC ; CCC(C)c1nc2c[nH]c(-c3c[nH]c4ccccc34)cn-2c1=O]

Under an argon atmosphere, 5-(1H-indol-3-yl)pyrazin-2-amine (40.0 mg, 0.19 mmol, 1 eq.) and 1,1-diethoxy-3-methylpentan-2-one (107 mg, 0.57 mmol, 2 eq.) were dissolved in ethanol (2 mL) and ultrapure water (Milli-Q) (0.2 mL) and then cooled to 0° C. The reaction solution was degassed in a vacuum, concentrated hydrochloric acid (0.1 mL) was added, and the mixture was stirred at 80° C. for 4 hours.

The mixture was cooled to room temperature and then concentrated under reduced pressure, and the residue was purified by column chromatography (eluent: chloroform/methanol=10/1) to obtain 2-(sec-butyl)-6-(1H-indol-3-yl)imidazo[1,2-a]pyrazin-3(7H)-one (CLA19) (19.3 mg, 33%).

¹H-NMR (500 MHz, CD₃OD): δ (ppm)=7.80 (t, J=5.9 Hz, 1H), 7.74 (s, 1H), 7.63 (s, 1H), 7.58 (s, 1H), 7.36 (d, J=8.0 Hz, 1H), 7.15-7.07 (m, 2H), 3.20 (quintet, J=3.2 Hz, 3H), 1.24 (d, J=6.9 Hz, 2H), 0.81 (t, J=7.4 Hz, 3H).

¹³C-NMR (125 MHz, CD₃OD): δ (ppm)=12.40, 19.02, 29.58, 35.36, 107.18, 113.08, 119.95, 121.72, 123.78, 125.50, 125.62, 138.49.

### Synthesis Example 3: Synthesis of 2-benzyl-6-(1H-pyrrolo[2,3-b]pyridin-3-yl)imidazo[1,2-a]pyrazin-3(7H)-one (CLA9)

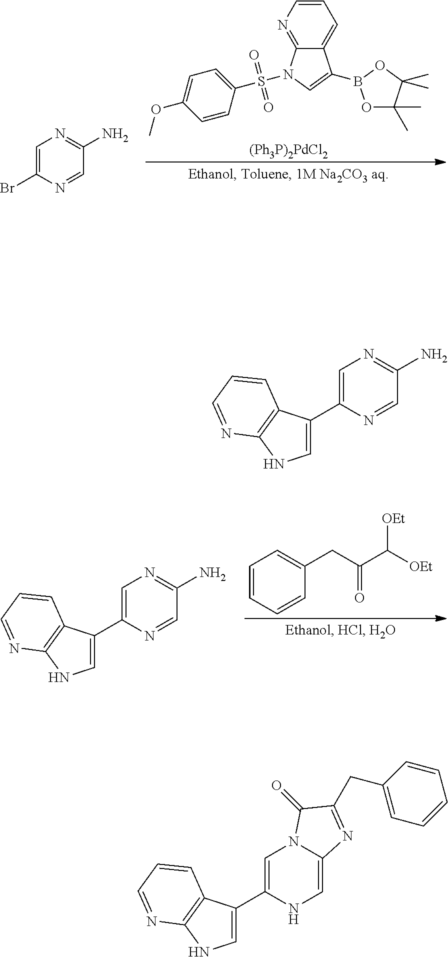

(1) Under an argon atmosphere, 5-bromopyrazin-2-amine (100.0 mg, 0.57 mmol, 1 eq.) and 1-((4-methoxyphenyl)sulfonyl)-3-(4,4,5,5-tetramethyl-1,3,2-dioxaborolan-2-yl)-1H-pyrrolo[2,3-b]pyridine (261.0 mg, 0.62 mmol, 1.1 eq.) were dissolved in ethanol (2 mL) and toluene (16 mL), a 1 M aqueous sodium carbonate solution (6 mL) was added thereto, and the mixture was stirred at room temperature. The reaction solution was degassed in a vacuum, tetrakis(triphenylphosphine) palladium(0) in a catalytic amount was added, and the mixture was degassed in a vacuum again and stirred at 100° C. overnight.

The mixture was cooled to room temperature, and the palladium catalyst was removed by celite filtration. The resulting residue was subjected to extraction with ethyl acetate, followed by washing with distilled water and saturated saline, and the resultant was dried over sodium sulfate and then concentrated under reduced pressure. The resulting residue was purified by column chromatography (eluent: hexane/ethyl acetate=4/1) and concentrated under reduced pressure. The resulting residue was dissolved in 1,4-dioxane (4 mL) and methanol (4 mL), a 5 N aqueous sodium hydroxide solution (3 mL) was added thereto, and the mixture was stirred at room temperature overnight. The reaction solution was subjected to extraction with ethyl acetate, followed by washing with distilled water and saturated saline, and the resultant was dried over sodium sulfate and then concentrated under reduced pressure. The resulting residue was purified by column chromatography (eluent: ethyl acetate/methanol=9/1) to obtain 5-(1H-pyrrole[2,3-b]pyridin-3-yl)pyrazin-2-amine (88.0 mg, 72%).

¹H-NMR (500 MHz, CD₃OD): δ (ppm)=8.40 (d, J=7.7 Hz, 1H), 8.24 (s, 1H), 8.17 (d, J=4.1 Hz, 1H), 7.98 (s, 1H), 7.66 (s, 1H), 7.10 (q, J=7.1 Hz, 1H).

¹³C-NMR (125 MHz, CD₃OD): δ (ppm)=112.54, 116.24, 118.34, 122.99, 129.77, 132.03, 137.90, 139.16, 142.52, 148.09, 152.62.
(2) Under an argon atmosphere, 5-(1H-pyrrole[2,3-b]pyridin-3-yl)pyrazin-2-amine (30.0 mg, 0.14 mmol, 1 eq.) obtained in (1) described above and 1,1-diethoxy-3-phenylpropan-2-one (63 mg, 0.28 mmol, 2 eq.) were dissolved in ethanol (2 mL) and Milli-Q (0.2 mL) and then cooled to 0° C. The reaction solution was degassed in a vacuum, concentrated hydrochloric acid (0.1 mL) was added, and the mixture was stirred at 80° C. for 4 hours.

The mixture was cooled to room temperature and then concentrated under reduced pressure, and the residue was purified by column chromatography (eluent: chloroform/methanol=4/1) to obtain 2-benzyl-6-(1H-pyrrolo[2,3-b]pyridin-3-yl)imidazo[1,2-a]pyrazin-3(7H)-one (CLA9) (31.7 mg, 65%).

¹H-NMR (500 MHz, CD₃OD): δ (ppm)=9.20 (q, J=3.0 Hz, 1H), 9.01 (br, 1H), 8.75 (br, 1H), 8.43 (q, J=2.2 Hz, 1H), 8.36 (s, 1H) 7.58 (q, J=4.6 Hz, 1H), 7.24-7.14 (m, 5H), 4.20 (s, 2H).

¹³C-NMR (125 MHz, CD₃OD): δ (ppm)=18.35, 30.84, 58.30, 112.36, 113.56, 118.00, 124.59, 128.29, 128.69, 129.64, 129.74, 130.00, 136.35, 136.57, 137.54, 137.78, 139.70, 140.02, 141.52.

### Synthesis Example 4: Synthesis of 2-(sec-butyl)-6-(4-(diethylamino)phenyl)imidazo[1,2-a]pyrazin-3(7H)-one (CLA21)

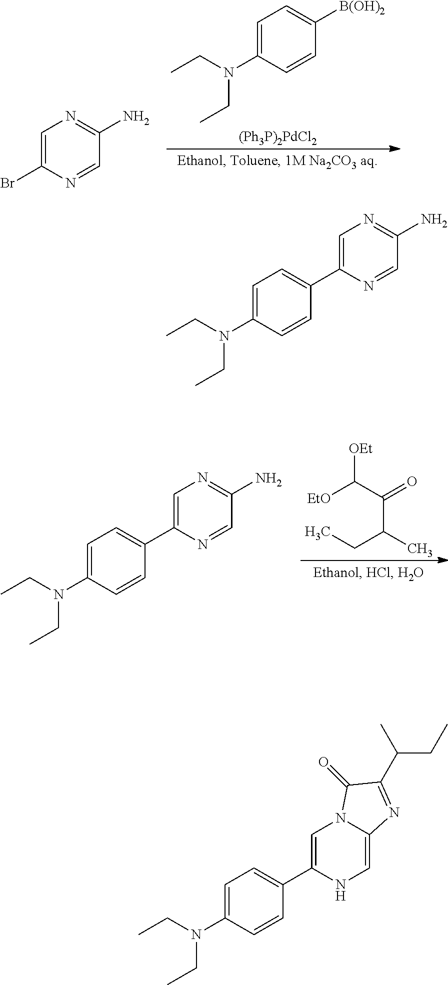

(1) Under an argon atmosphere, 5-bromopyrazin-2-amine (200.0 mg, 1.14 mmol, 1 eq.) and (4-(diethylamino)phenyl)boronic acid (355.0 mg, 1.83 mmol, 1.6 eq.) were dissolved in ethanol (3.5 mL) and toluene (24 mL), a 1 M aqueous sodium carbonate solution (9 mL) was then added thereto, and the mixture was stirred at room temperature. The reaction solution was degassed in a vacuum, tetrakis(triphenylphosphine) palladium(0) in a catalytic amount was added, and the mixture was degassed in a vacuum again and stirred at 100° C. overnight.

The mixture was cooled to room temperature, and the palladium catalyst was removed by celite filtration. The resulting residue was subjected to extraction with ethyl acetate, followed by washing with distilled water and saturated saline, and the resultant was dried over sodium sulfate and then concentrated under reduced pressure. The resulting residue was purified by column chromatography (eluent: hexane/ethyl acetate=1/1) to obtain 5-(4-(diethylamino)phenyl)pyrazin-2-amine (261.9 mg, 94%).

¹H-NMR (500 MHz, CDCl₃): δ (ppm)=8.36 (d, J=1.4 Hz, 1H), 8.00 (d, J=1.5 Hz, 1H), 7.74 (q, J=3.0 Hz, 2H), 6.74 (d, J=8.7 Hz, 2H), 3.39 (q, J=7.0 Hz, 4H), 1.18 (t, J=7.0 Hz, 6H).

¹³C-NMR (125 MHz, CDCl₃): δ (ppm)=12.68, 44.56, 111.98, 124.16, 126.83, 131.38, 137.86, 143.90, 147.80, 152.14.
(2) Under an argon atmosphere, 5-(4-(diethylamino)phenyl)pyrazin-2-amine (40.0 mg, 0.16 mmol, 1 eq.) obtained in (1) described above and 1,1-diethoxy-3-methylpentan-2-one (93 mg, 0.49 mmol, 3 eq.) were dissolved in ethanol (2 mL) and ultrapure water (Milli-Q) (0.2 mL) and then cooled to 0° C. The reaction solution was degassed in a vacuum, concentrated hydrochloric acid (0.1 mL) was added, and the mixture was stirred at 80° C. for 4 hours.

The mixture was cooled to room temperature and then concentrated under reduced pressure, and the residue was purified by column chromatography (eluent: chloroform/methanol=20/1) to obtain 2-(sec-butyl)-6-(4-(diethylamino)phenyl)imidazo[1,2-a]pyrazin-3(7H)-one (CLA21) (38.9 mg, 70%).

¹H-NMR (500 MHz, CD₃OD): δ (ppm)=8.98 (s, 1H), 8.75 (s, 1H), 8.23 (d, J=8.6 Hz, 2H), 7.75 (d, J=8.4 Hz, 2H), 3.64 (q, J=7.2 Hz, 4H), 1.77-1.69 (m, 2H), 1.33 (d, J=6.9 Hz, 3H), 1.11 (t, J=7.2 Hz, 6H), 0.85 (t, J=7.3 Hz, 3H).

¹³C-NMR (125 MHz, CD₃OD): δ (ppm)=10.78, 12.25, 18.36, 19.46, 29.97, 33.09, 54.66, 58.29, 113.28, 124.32, 129.21, 129.97, 135.74, 137.55, 139.69.

### Synthesis Example 5: Synthesis of 1-(3-(2-benzyl-6-(4-(diethylamino)phenyl)-3-oxo-3,7-dihydroimidazo[1,2-a]pyrazin-8-yl)propyl)guanidine (CLA12)

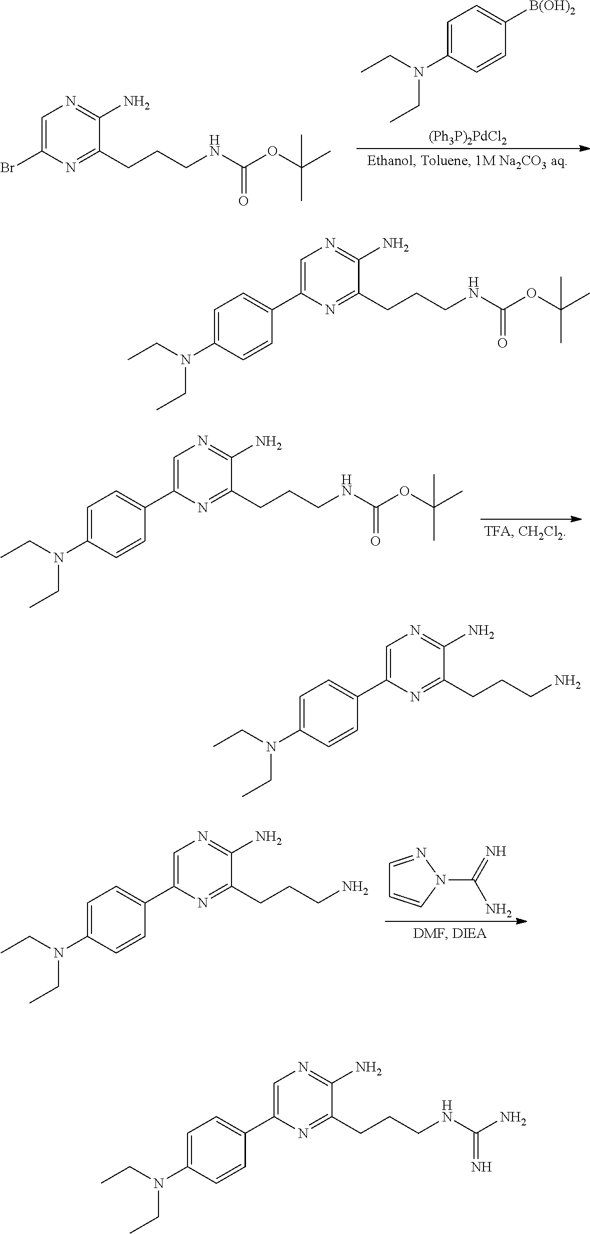

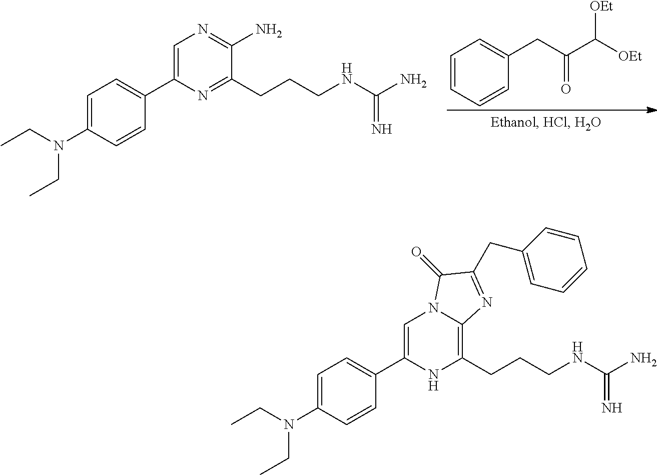 [SMILES: CCN(CC)c1ccc(-c2cnc(N)c(CCCNC(=N)N)n2)cc1 ; CCOC(OCC)C(=O)Cc1ccccc1 ; CCN(CC)c1ccc(-c2cn3c(=O)c(Cc4ccccc4)nc-3c(CCCNC(=N)N)[nH]2)cc1]

(1) Under an argon atmosphere, tert-butyl(3-(3-amino-6-bromopyrazin-2-yl)propyl)carbamate (150.0 mg, 0.45 mmol, 1 eq.) and (4-(diethylamino)phenyl)boronic acid (122.3 mg, 0.63 mmol, 1.4 eq.) were dissolved in ethanol (3 mL) and toluene (24 mL), a 1 M aqueous sodium carbonate solution (9 mL) was then added thereto, and the mixture was stirred at room temperature. The reaction solution was degassed in a vacuum, tetrakis(triphenylphosphine) palladium(0) in a catalytic amount was added, and the mixture was degassed in a vacuum again and stirred at 100° C. overnight.

The mixture was cooled to room temperature, and the palladium catalyst was removed by celite filtration. The resulting residue was subjected to extraction with ethyl acetate, followed by washing with distilled water and saturated saline, and the resultant was dried over sodium sulfate and then concentrated under reduced pressure. The resulting residue was purified by column chromatography (eluent: hexane/ethyl acetate=2/3) to obtain tert-butyl(3-(3-amino-6-(4-(diethylamino)phenyl)pyrazin-2-yl)propyl)carbamate (136.9 mg, 75%).

¹H-NMR (500 MHz, CDCl₃): δ (ppm)=8.15 (s, 1H), 7.68 (d, J=8.9 Hz, 2H), 6.65 (d, J=8.8 Hz, 2H), 4.90 (s, 1H), 4.43 (s, 2H), 3.31 (q, J=3.3 Hz, 4H), 3.18 (q, J=6.2 Hz, 2H), 2.64 (t, J=7.2 Hz, 2H), 1.99 (quintet, J=6.9 Hz, 2H), 1.36 (s, 9H), 1.10 (t, J=7.0 Hz, 6H).

¹³C-NMR (125 MHz, CDCl₃): δ (ppm)=12.62, 26.38, 28.43, 30.23, 40.24, 44.47, 79.09, 111.89, 124.48, 126.75, 135.34, 140.61, 143.27, 147.66, 150.15, 156.22.
(2) Under an argon atmosphere, tert-butyl(3-(3-amino-6-(4-(diethylamino)phenyl)pyrazin-2-yl)propyl)carbamate (135.6 mg, 0.33 mmol, 1 eq.) obtained in (1) described above was dissolved in TFA (1 mL) and methylene chloride (6 mL), and the mixture was stirred at room temperature for 4 hours. The reaction solution was subjected to extraction with ethyl acetate, followed by washing with distilled water and saturated saline, and the resultant was dried over sodium sulfate and then concentrated under reduced pressure. As a result, 3-(3-aminopropyl)-5-(4-(diethylamino)phenyl)pyrazin-2-amine was obtained. The product was used in the subsequent reaction without further purification.
(3) Under an argon atmosphere, 3-(3-aminopropyl)-5-(4-(diethylamino)phenyl)pyrazin-2-amine (375.0 mg, 1.25 mmol, 1 eq.) obtained in (2) described above and 1H-pyrazole-1-carboxamidine hydrochloride (369.0 mg, 2.51 mmol, 2 eq.) were dissolved in DMF (18 mL), DIEA (621 mg) was then added thereto, and the mixture was stirred at room temperature overnight.

Ether was added, resulting in precipitation, and the precipitate was washed with an acetone-ethanol mixed solution (acetone/ethanol=4/1) (10 mL) to obtain 1-(3-(3-amino-6-(4-(diethylamino)phenyl)pyrazin-2-yl)propyl)guanidine (461.6 mg, quant.).

The product was used in the subsequent reaction without further purification.
(4) Under an argon atmosphere, 1-(3-(3-amino-6-(4-(diethylamino)phenyl)pyrazin-2-yl)propyl)guanidine (100.0 mg, 0.29 mmol, 1 eq.) obtained in (3) described above and 1,1-diethoxy-3-phenylpropan-2-one (104.0 mg, 0.46 mmol, 2 eq.) were dissolved in ethanol (4 mL) and ultrapure water (Milli-Q) (0.4 mL) and then cooled to 0° C.

The reaction solution was degassed in a vacuum, concentrated hydrochloric acid (0.2 mL) was added, and the mixture was stirred at 80° C. for 4 hours. The mixture was cooled to room temperature and then concentrated under reduced pressure, and the residue was purified by column chromatography (eluent: ethanol/water=2/1) to obtain 1-(3-(2-benzyl-6-(4-(diethylamino)phenyl)-3-oxo-3,7-dihydroimidazo[1,2-a]pyrazin-8-yl)propyl)guanidine (CLA12) (30.4 mg, 22%).

¹H-NMR (500 MHz, CD₃OD): δ (ppm)=8.97 (s, 1H), 8.44 (br, 2H), 7.90 (br, 2H), 7.38-7.23 (m, 5H), 4.38 (s, 2H), 3.77 (s, 4H), 3.46 (br, 2H), 2.35 (br, 2H), 1.29-1.17 (m, 9H).

¹³C-NMR (125 MHz, CD₃OD): δ (ppm)=10.88, 18.33, 26.74, 30.26, 30.87, 41.95, 55.10, 58.28, 112.81, 124.69, 128.18, 128.30, 129.66, 129.94, 130.26, 137.85, 138.51, 138.93, 139.31, 141.12, 149.89, 158.62.

### Synthesis Example 6: Synthesis of 1-(3-(6-(4-(diethylamino)phenyl)-2-methyl-3-oxo-3,7-dihydroimidazo[1,2-a]pyrazin-8-yl)propyl)guanidine (CLA13)

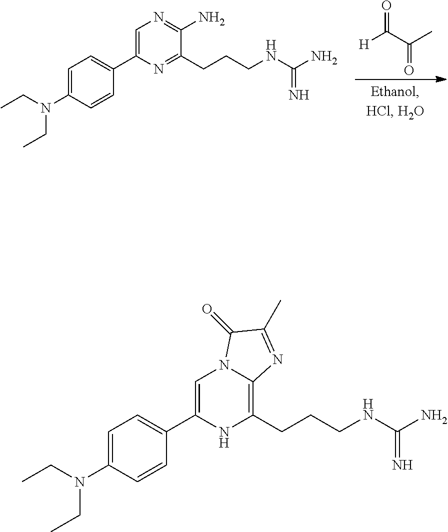

In the same manner as in (1) to (3) of Synthesis Example 5, 1-(3-(3-amino-6-(4-(diethylamino)phenyl)pyrazin-2-yl)propyl)guanidine was obtained.

Next, under an argon atmosphere, 1-(3-(3-amino-6-(4-(diethylamino)phenyl)pyrazin-2-yl)propyl)guanidine (100.0 mg, 0.29 mmol, 1 eq.) and methylglyoxal (42.0 mg, 0.58 mmol, 2 eq.) were dissolved in ethanol (4 mL) and ultrapure water (Milli-Q) (0.4 mL) and then cooled to 0° C. The reaction solution was degassed in a vacuum, concentrated hydrochloric acid (0.2 mL) was added, and the mixture was stirred at 80° C. for 5 hours.

The mixture was cooled to room temperature and then concentrated under reduced pressure, and the residue was purified by column chromatography (eluent: ethanol/water=1/4) to obtain 1-(3-(6-(4-(diethylamino)phenyl)-2-methyl-3-oxo-3,7-dihydroimidazo[1,2-a]pyrazin-8-yl)propyl)guanidine (CLA13) (22.1 mg, 19%).

¹H-NMR (500 MHz, CD₃OD): δ (ppm)=8.79 (s, 1H), 8.31 (d, J=8.5 Hz, 2H), 7.75 (d, J=8.3 Hz, 2H), 3.65 (q, J=6.6 Hz, 5H), 3.50 (q, J=7.0 Hz, 5H), 3.20 (quintet, J=1.6 Hz, 2H), 2.48 (s, 3H), 2.36-2.28 (m, 2H), 1.37 (br, 2H).

### Synthesis Example 7: Synthesis of 1-(3-(6-(4-(diethylamino)phenyl)-2-(4-hydroxybenzyl)-3-oxo-3,7-dihydroimidazo[1,2-a]pyrazin-8-yl)propyl)guanidine (CLA14)

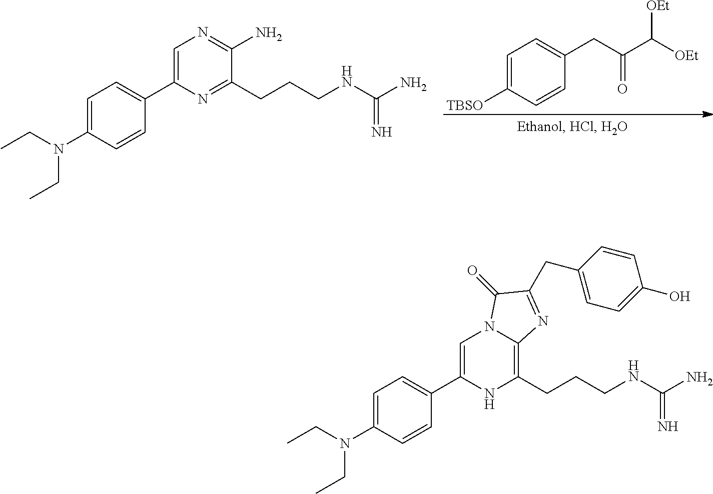 [SMILES: CCN(CC)c1ccc(-c2cnc(N)c(CCCNC(=N)N)n2)cc1 ; CCOC(OCC)C(=O)Cc1ccc(O[Si](C)(C)C(C)(C)C)cc1 ; CCN(CC)c1ccc(-c2cn3c(=O)c(Cc4ccc(O)cc4)nc-3c(CCCNC(=N)N)[nH]2)cc1]

In the same manner as in (1) to (3) of Synthesis Example 5, 1-(3-(3-amino-6-(4-(diethylamino)phenyl)pyrazin-2-yl)propyl)guanidine was obtained.

Next, under an argon atmosphere, 1-(3-(3-amino-6-(4-(diethylamino)phenyl)pyrazin-2-yl)propyl)guanidine (100.0 mg, 0.29 mmol, 1 eq.) and 3-(4-((tert-butyldimethylsilyl)oxy)phenyl)-1,1-diethoxypropan-2-one (162.0 mg, 0.46 mmol, 1.6 eq.) were dissolved in ethanol (4 mL) and ultrapure water (Milli-Q) (0.4 mL) and then cooled to 0° C. The reaction solution was degassed in a vacuum, concentrated hydrochloric acid (0.2 mL) was added, and the mixture was stirred at 80° C. for 4 hours.

The mixture was cooled to room temperature and then concentrated under reduced pressure, and the residue was purified by column chromatography (eluent: ethanol/water=1/4) to obtain 1-(3-6-(4-(diethylamino)phenyl)-2-(4-hydroxybenzyl)-3-oxo-3,7-dihydroimidazo[1,2-a]pyrazin-8-yl)propyl)guanidine (CLA14) (41.5 mg, 29%).

¹H-NMR (500 MHz, CD₃OD): δ (ppm)=8.81 (s, 1H), 8.31 (d, J=6.9 Hz, 2H), 7.77 (d, J=6.1 Hz, 2H), 7.07 (d, J=7.5 Hz, 2H), 6.64 (d, J=7.3 Hz, 2H), 4.13 (s, 2H), 3.64 (br, 5H), 3.50 (q, J=7.0 Hz, 5H), 3.26 (br, 2H), 3.20 (quintet, J=1.5 Hz, 2H), 2.21 (br, 2H).

### Synthesis Example 8: Synthesis of 1-(3-(2-(sec-butyl)-6-(4-(diethylamino)phenyl)-3-oxo-3,7-dihydroimidazo[1,2-a]pyrazin-8-yl)propyl)guanidine (CLA20)

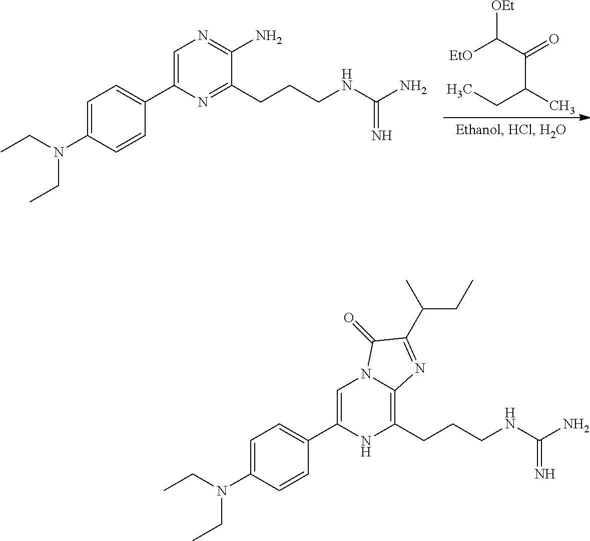 [SMILES: CCN(CC)c1ccc(-c2cnc(N)c(CCCNC(=N)N)n2)cc1 ; CCOC(OCC)C(=O)C(C)CC ; CCC(C)c1nc2c(CCCNC(=N)N)[nH]c(-c3ccc(N(CC)CC)cc3)cn-2c1=O]

In the same manner as in (1) to (3) of Synthesis Example 5, 1-(3-(3-amino-6-(4-(diethyl amino)phenyl)pyrazin-2-yl)propyl)guanidine was obtained.

Next, under an argon atmosphere, 1-(3-(3-amino-6-(4-(diethylamino)phenyl)pyrazin-2-yl)propyl)guanidine (100.0 mg, 0.29 mmol, 1 eq.) and 1,1-diethoxy-3-methylpentan-2-one (110.0 mg, 0.58 mmol, 2 eq.) were dissolved in ethanol (4 mL) and ultrapure water (Milli-Q) (0.4 mL) and then cooled to 0° C. The reaction solution was degassed in a vacuum, concentrated hydrochloric acid (0.2 mL) was added, and the mixture was stirred at 80° C. for 4 hours.

The mixture was cooled to room temperature and then concentrated under reduced pressure, and the residue was purified by column chromatography (eluent: ethanol/water=1/4) to obtain 1-(3-(2-(sec-butyl)-6-(4-(diethylamino)phenyl)-3-oxo-3,7-dihydroimidazo[1, 2-a]pyrazin-8-yl)propyl)guanidine (CLA20) (61.2 mg, 47%).

¹H-NMR (500 MHz, CD₃OD): δ (ppm)=8.96 (s, 1H), 8.45 (d, J=6.3 Hz, 2H), 7.93 (d, J=5.5 Hz, 2H), 3.77 (br, 4H), 3.50 (br, 4H), 2.37 (s, 2H), 1.98-1.87 (m, 2H), 1.51 (d, J=6.6 Hz, 3H), 1.23 (br, 6H), 0.98 (d, J=6.8 Hz, 3H).

¹³C-NMR (125 MHz, CD₃OD): δ (ppm)=10.78, 12.50, 19.64, 26.80, 29.86, 30.97, 32.92, 41.87, 49.84, 54.96, 112.43, 124.65, 127.02, 128.29, 130.08, 137.53, 138.54, 139.29, 141.22, 149.84, 158.64.

### Synthesis Example 9: Synthesis of 1-(3-(2-(4-hydroxybenzyl)-6-(1H-indol-3-yl)-3-oxo-3,7-dihydroimidazo[1,2-a]pyrazin-8-polypropyl)guanidine (CLA15)

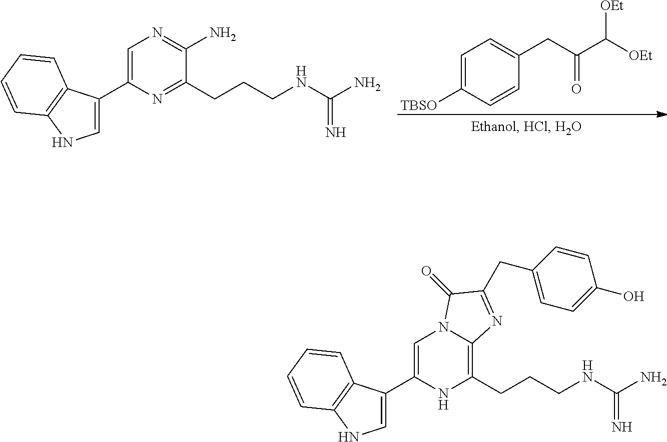 [SMILES: N=C(N)NCCCc1nc(-c2c[nH]c3ccccc23)cnc1N ; CCOC(OCC)C(=O)Cc1ccc(O[Si](C)(C)C(C)(C)C)cc1 ; N=C(N)NCCCc1[nH]c(-c2c[nH]c3ccccc23)cn2c(=O)c(Cc3ccc(O)cc3)nc1-2]

Under an argon atmosphere, 1-(3-(3-amino-6-(1H-indol-3-yl)pyrazin-2-yl)propyl)guanidine (100.0 mg, 0.32 mmol, 1 eq.) and 3-(4-((tert-butyldimethylsilyl)oxy)phenyl)-1,1-diethoxypropan-2-one (182.0 mg, 0.51 mmol, 1.6 eq.) were dissolved in ethanol (4 mL) and ultrapure water (Milli-Q) (0.4 mL) and then cooled to 0° C. The reaction solution was degassed in a vacuum, concentrated hydrochloric acid (0.2 mL) was added, and the mixture was stirred at 80° C. for 4 hours.

The mixture was cooled to room temperature and then concentrated under reduced pressure, and the residue was purified by column chromatography (eluent: ethanol/water=1/2) to obtain 1-(3-(2-(4-hydroxybenzyl)-6-(1H-indol-3-yl)-3-oxo-3,7-dihydroimidazo[1,2-a]pyrazin-8-yl)propyl)guanidine (CLA15) (45.3 mg, 30%).

¹H-NMR (500 MHz, CD₃OD): δ (ppm)=7.73 (s, 1H), 7.63 (s, 1H), 7.52 (d, J=7.6 Hz, 1H), 7.14 (d, J=8.3 Hz, 2H), 7.00 (d, J=7.8 Hz, 1H), 6.72-6.63 (m, 4H), 3.50 (q, J=7.0 Hz, 2H), 3.27 (t, J=6.5 Hz, 2H), 2.82 (br, 2H), 1.98 (br, 2H).

### Synthesis Example 10: 2-benzyl-6-(4-(diethylamino)phenyl)-8-(2-(pyridin-4-yl)ethyl)imidazo[1,2-a]pyrazin-3(7H)-one (CLA18)

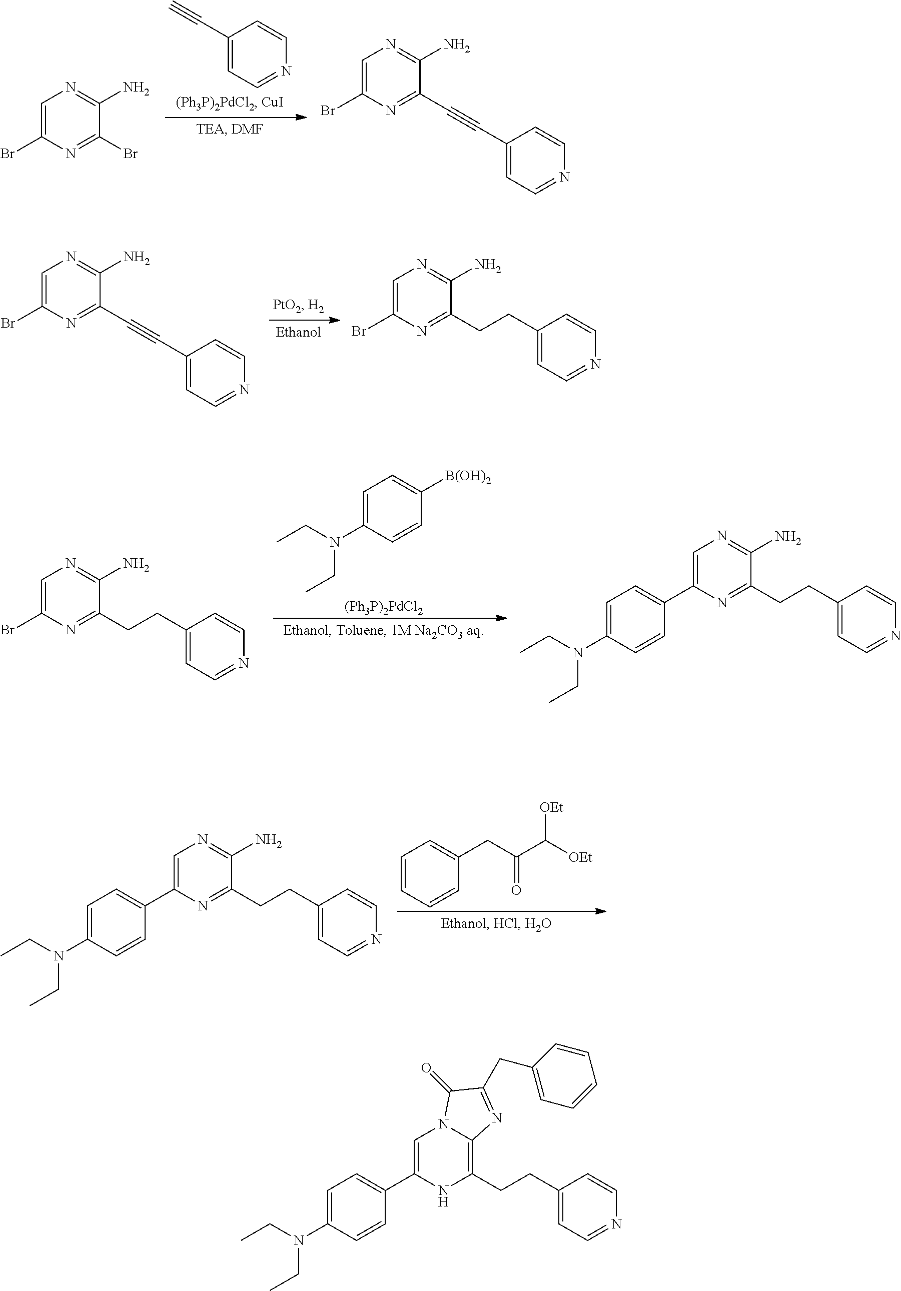

(1) Under an argon atmosphere, 3,5-dibromopyrazin-2-amine (1,000.0 mg, 3.95 mmol, 1 eq.) and 4-ethynylpyridine (448.5 mg, 4.34 mmol, 1.1 eq.) were dissolved in DMF (30 mL), TEA (15 mL) was then added thereto, and the mixture was stirred at room temperature. The reaction solution was degassed in a vacuum, tetrakis(triphenylphosphine) palladium(0) and copper chloride in a catalytic amount were added, and the mixture was degassed in a vacuum again and stirred at 120° C. for 40 minutes.

The mixture was cooled to room temperature, and the catalyst was removed by celite filtration. The resulting residue was subjected to extraction with ethyl acetate, followed by washing with distilled water and saturated saline, and the resultant was dried over sodium sulfate and then concentrated under reduced pressure. The resulting residue was purified by column chromatography (eluent: ethyl acetate/methanol=10/1) to obtain 5-buromo-3-(pyridin-4-ylethynyl)pyrazin-2-amine (748 mg, 68%).

¹H-NMR (500 MHz, CD₃OD, CDCl₃): δ (ppm)=8.50 (q, J=2.0 Hz, 2H), 7.99 (s, 1H), 7.48 (q, J=2.0 Hz, 2H), 4.41 (s, 2H).

¹³C-NMR (125 MHz, CD₃OD, CDCl₃): δ (ppm)=87.70, 93.00, 122.39, 124.66, 125.99, 130.52, 144.99, 149.17, 155.18.
(2) Under an argon atmosphere, 5-bromo-3-(pyridin-4-ylethynyl)pyrazin-2-amine (720 mg, 2.61 mmol, 1 eq.) obtained in (1) described above was dissolved in ethanol (25 mL), platinum oxide in a catalytic amount was added, and the reaction solution was degassed in a vacuum and then stirred at room temperature overnight. The catalyst was removed by celite filtration. The resulting residue was purified by column chromatography (eluent: ethyl acetate/methanol=10/1) to obtain 5-bromo-3-(2-(pyridin-4-yl)ethyl)pyrazin-2-amine (470 mg, 64%).

¹H-NMR (500 MHz, CDCl₃): δ (ppm)=8.42 (q, J=1.9 Hz, 2H), 7.91 (s, 1H), 7.09 (q, J=1.9 Hz, 2H), 4.52 (s, 2H), 3.06 (t, J=7.8 Hz, 2H), 2.81 (t, J=7.8 Hz, 2H).

¹³C-NMR (125 MHz, CDCl₃): δ (ppm)=31.47, 33.32, 124.02, 126.77, 141.40, 142.17, 149.96, 150.15, 151.66.
(3) Under an argon atmosphere, 5-bromo-3-(2-(pyridin-4-yl)ethyl)pyrazin-2-amine (200.0 mg, 0.71 mmol, 1 eq.) obtained in (2) described above and (4-(diethylamino)phenyl)boronic acid (193.0 mg, 0.85 mmol, 1.2 eq.) were dissolved in ethanol (3 mL) and toluene (20 mL), a 1 M aqueous sodium carbonate solution (8 mL) was then added thereto, and the mixture was stirred at room temperature. The reaction solution was degassed in a vacuum, tetrakis(triphenylphosphine) palladium(0) in a catalytic amount was added, and the mixture was degassed in a vacuum again and stirred at 100° C. for 5 hours.

The mixture was cooled to room temperature, and the palladium catalyst was removed by celite filtration. The resulting residue was subjected to extraction with ethyl acetate, followed by washing with distilled water and saturated saline, and the resultant was dried over sodium sulfate and then concentrated under reduced pressure. The resulting residue was purified by column chromatography (eluent: ethyl acetate/methanol=10/1) to obtain 5-(4-(diethylamino)phenyl)-3-(2-(pyridin-4-yl)ethyl)pyrazin-2-amine (224 mg, 90%).
(4) Under an argon atmosphere, 5-(4-(diethylamino)phenyl)-3-(2-(pyridin-4-yl)ethyl)pyrazin-2-amine (60.0 mg, 0.17 mmol, 1 eq.) obtained in (3) described above and 1,1-diethoxy-3-phenylpropan-2-one (76.0 mg, 0.34 mmol, 2 eq.) were dissolved in ethanol (4 mL) and Milli-Q (0.4 mL) and then cooled to 0° C. The reaction solution was degassed in a vacuum, concentrated hydrochloric acid (0.2 mL) was added, and the mixture was stirred at 80° C. for 4 hours.

The mixture was cooled to room temperature and then concentrated under reduced pressure, and the residue was purified by chromatography (eluent: chloroform/methanol=5/1) to obtain 2-benzyl-6-(4-(diethylamino)phenyl)-8-(2-(pyridin-4-yl)ethyl)imidazo[1,2-a]pyrazin-3(7H)-one (CLA18) (71.2 mg, 86%).

¹H-NMR (500 MHz, CD₃OD): δ (ppm)=8.65 (d, J=5.8 Hz, 2H), 8.62 (s, 1H), 8.14 (d, J=8.7 Hz, 2H), 8.06 (d, J=5.6 Hz, 2H), 7.73 (br, 2H), 7.22-7.07 (m, 5H), 4.17 (s, 2H), 3.67-3.59 (m, 8H), 1.09 (t, J=7.1 Hz, 6H).

¹³C-NMR (125 MHz, CD₃OD): δ (ppm)=10.84, 18.38, 30.89, 31.99, 33.18, 49.84, 54.49, 58.28, 112.05, 124.10, 127.84, 127.96, 128.79, 129.57, 129.76, 129.78, 137.70, 138.53, 139.64, 142.11, 147.37, 164.77.

### Synthesis Example 11: Synthesis of 2-benzyl-6-(4-(diphenylamino)phenyl)-8-(2-(pyridin-4-yl)ethyl)imidazo[1,2-a]pyrazin-3(7H)-one (CLA17)

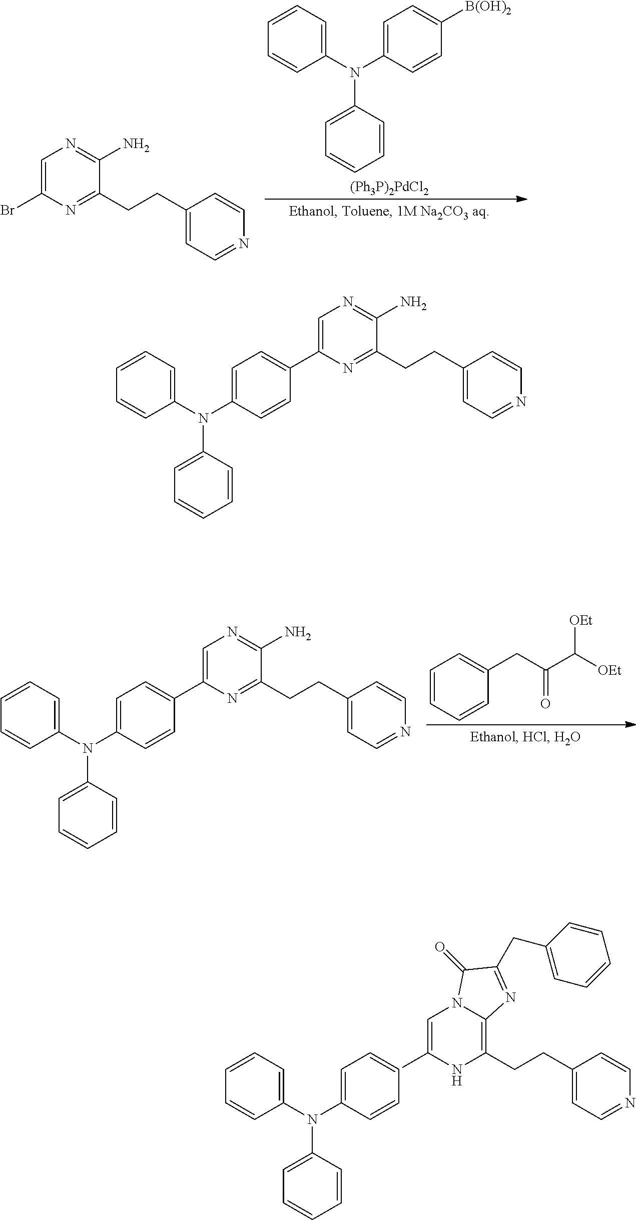

(1) Under an argon atmosphere, 5-bromo-3-(2-(pyridin-4-yl)ethyl)pyrazin-2-amine (200.0 mg, 0.71 mmol, 1 eq.) and (4-(diphenylamino)phenyl)boronic acid (245.0 mg, 0.85 mmol, 1.2 eq.) were dissolved in ethanol (3 mL) and toluene (20 mL), a 1 M aqueous sodium carbonate solution (8 mL) was then added thereto, and the mixture was stirred at room temperature. The reaction solution was degassed in a vacuum, tetrakis(triphenylphosphine) palladium(0) in a catalytic amount was added, and the mixture was degassed in a vacuum again and stirred at 100° C. for 4 hours.

The mixture was cooled to room temperature, and the palladium catalyst was removed by celite filtration. The resulting residue was subjected to extraction with ethyl acetate, followed by washing with distilled water and saturated saline, and the resultant was dried over sodium sulfate and then concentrated under reduced pressure. The resulting residue was purified by column chromatography (eluent: ethyl acetate/methanol=10/1) to obtain 5-(4-(diphenylamino)phenyl)-3-(2-(pyridin-4-yl)ethyl)pyrazin-2-amine (302 mg, 95%).
(2) Under an argon atmosphere, 5-(4-(diphenylamino)phenyl)-3-(2-(pyridin-4-yl)ethyl)pyrazin-2-amine (60.0 mg, 0.13 mmol, 1 eq.) obtained in (1) described above and 1,1-diethoxy-3-phenylpropan-2-one (60.0 mg, 0.27 mmol, 2 eq.) were dissolved in ethanol (4 mL) and Milli-Q (0.4 mL) and then cooled to 0° C. The reaction solution was degassed in a vacuum, concentrated hydrochloric acid (0.2 mL) was added, and the mixture was stirred at 80° C. for 4 hours.

The mixture was cooled to room temperature and then concentrated under reduced pressure, and the residue was purified by chromatography (eluent: chloroform/methanol=10/1) to obtain 2-benzyl-6-(4-(diphenylamino)phenyl)-8-(2-(pyridin-4-yl)ethyl)imidazo[1,2-a]pyrazin-3(7H)-one (CLA17) (42.1 mg, 54%).

¹H-NMR (500 MHz, CD₃OD): δ (ppm)=8.62 (d, J=6.4 Hz, 2H), 8.42 (s, 1H), 8.02 (d, J=6.2 Hz, 2H), 7.70 (d, J=8.4 Hz, 2H), 7.24-6.94 (m, 17H), 4.18 (s, 2H), 3.64 (t, J=6.8 Hz, 2H), 3.57 (t, J=6.9 Hz, 2H).

¹³C-NMR (125 MHz, CD₃OD): δ (ppm)=18.36, 30.67, 31.92, 33.10, 49.82, 58.29, 110.07, 123.19, 125.05, 126.30, 127.73, 128.10, 128.41, 128.63, 128.78, 129.60, 129.87, 130.61, 138.17, 142.07, 147.01, 148.46, 150.86, 164.72.

### Synthesis Example 12: Synthesis of 6-(1H-indol-3-yl)-2-methylimidazo[1,2-a]pyrazin-3(7H)-one (CLA2)

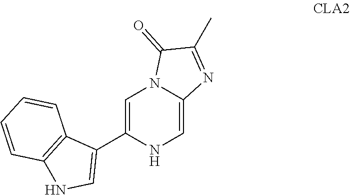 [SMILES: Cc1nc2c[nH]c(-c3c[nH]c4ccccc34)cn-2c1=O]

A target compound was synthesized in accordance with the description of Literature: Photochem. Photobiol. Sci., 2008, 7, pp. 197 to 207.

### Synthesis Example 13: Synthesis of 2-benzyl-6-(4-(diethylamino)phenyl)imidazo[1,2-a]pyrazin-3(7H)-one (CLA3)

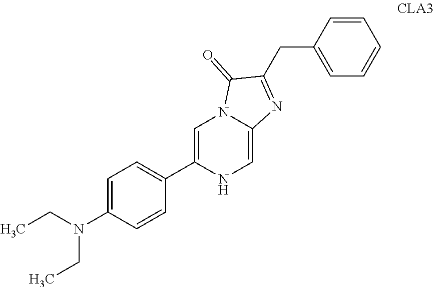 [SMILES: CCN(CC)c1ccc(-c2cn3c(=O)c(Cc4ccccc4)nc-3c[nH]2)cc1]

A target compound was synthesized in accordance with the description of WO2021/187531.

### Synthesis Example 14: Synthesis of 6-(4-(diethylamino)phenyl)-2-methylimidazo[1,2-a]pyrazin-3(7H)-one (CLA4)

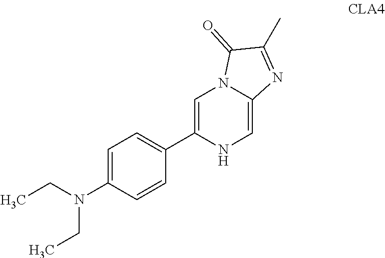 [SMILES: CCN(CC)c1ccc(-c2cn3c(=O)c(C)nc-3c[nH]2)cc1]

A target compound was synthesized in accordance with the description of WO2021/187531.

### Synthesis Example 15: Synthesis of 2-benzyl-6-(4-(dimethylamino)phenyl)imidazo[1,2-a]pyrazin-3(7H)-one (CLA7)

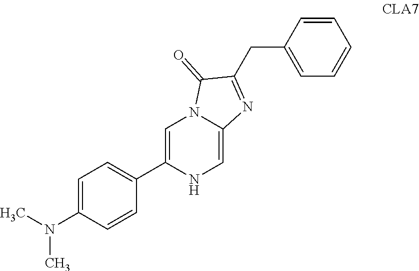 [SMILES: CN(C)c1ccc(-c2cn3c(=O)c(Cc4ccccc4)nc-3c[nH]2)cc1]

A target compound was synthesized in accordance with the description of WO2021/187531.

### Synthesis Example 16: Synthesis of 6-(4-(dimethylamino)phenyl)-2-methylimidazo[1,2-a]pyrazin-3(7H)-one (CLA8)

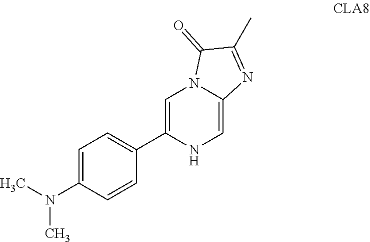

A target compound was synthesized in accordance with the description of WO2021/187531.

### Synthesis Example 17: Synthesis of 2-benzyl-6-(4-(pyrrolidin-1-yl)phenyl)imidazo[1,2-a]pyrazin-3(7H)-one (CLA10)

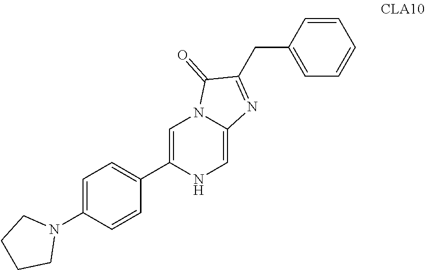 [SMILES: O=c1c(Cc2ccccc2)nc2c[nH]c(-c3ccc(N4CCCC4)cc3)cn1-2]

A target compound was synthesized in accordance with the description of WO2021/187531.

### Synthesis Example 18: Synthesis of 1-(3-(6-(4-(diethylamino)phenyl)-2-methyl-3-oxo-3, 7-dihydroimidazo[1,2-a]pyrazin-8-yl)propyl)guanidine (CLA16)

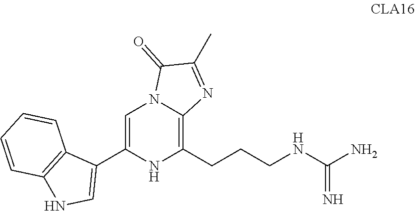 [SMILES: Cc1nc2c(CCCNC(=N)N)[nH]c(-c3c[nH]c4ccccc34)cn-2c1=O]

A target compound was synthesized in accordance with the description of JP 2012-95649 A.

### 2. Example 1

Each of native Cypridina luciferin (Product number: 3512055 manufactured by ATTO Corporation) and the Cypridina luciferin derivative obtained in each of Synthesis Examples was allowed to react with a spike protein derived from a SARS-CoV-2 virus, and luminescence was measured.

### (Measurement Condition)

In a 96-well plate (½ Area OpticalPlate-96, manufactured by PerkinElmer, Inc.), 5 μL of a protein solution (936 μg/mL) and 45 L of a luciferin-buffer solution (20 μM luciferin shown in Table 1, 20 mM HEPES, pH: 7.4) were mixed, and a signal was immediately measured for 1 minute using a luminescence plate reader (GloMax Explorer Multimode Microplate Reader manufactured by Promega Corporation) to determine the luminescence intensity (Luminescence) ([RLU/min]) (n=3).

As the protein solution, a spike protein derived from a SARS-CoV-2 virus (Trimeric SARS-CoV-2 Spike Protein, Full-length, BSV-COV-PR-34, manufactured by M&S TechnoSystems Inc.; purified protein solution) was used. It is known that the coronavirus spike protein generally forms a membrane-bound trimer on the viral envelope, and the trimer was used.

As a control protein solution, a solution of human serum-derived IgA (Product number: 306-51123, manufactured by FUJIFILM Wako Pure Chemical Corporation) was used, and a signal-to-noise ratio (S/N ratio) was calculated. The results are shown in Table 1.

| Compound | S/N ratio |
|---|---|
| Cypridina Luciferin | 28.1 |
| CLA1 | 6.63 |
| CLA2 | 5.81 |
| CLA3 | 6.77 |
| CLA4 | 6.87 |
| CLA7 | 5.15 |
| CLA8 | 4.04 |
| CLA9 | 4.05 |
| CLA10 | 6.76 |
| CLA12 | 6.33 |
| CLA13 | 5.12 |
| CLA14 | 8.22 |
| CLA15 | 15.9 |
| CLA16 | 6.81 |
| CLA17 | 1.95 |
| CLA18 | 4.46 |
| CLA19 | 6.78 |
| CLA20 | 10.6 |
| CLA21 | 7.06 |

### 3. Example 2

In order to specify a luminescent reaction field in a spike protein of native Cypridina luciferin for the purpose of specifying the luminescent reaction field of native Cypridina luciferin in a spike protein of a SARS-CoV-2 virus, the luminescence activity between each domain of the SARS-CoV-2 spike protein and native Cypridina luciferin was investigated in addition to the whole length of the SARS-CoV-2 spike protein.

The SARS coronavirus spike protein is divided into three domains: S1, S2, and a receptor-binding domain (RBD). As protein samples, the following samples were used. All the samples were manufactured by Sino Biological, Inc., and were monomers. Moreover, the samples were provided in a freeze-dried state.
SARS-CoV2 Spike S1+S2 (Product number: 40589-V08H04)
SARS-CoV2 Spike S1 (Product number: 40591-V08)
SARS-CoV2 Spike S2 (Product number: 40590-V08H1)
SARS-CoV2 Spike RBD (Product number: 40592-V08H)
SARS-CoV2 Spike RBD (Y453F) (Product number: 40592-V08H80)
SARS-CoV2 Spike RBD (N501Y) (Product number: 40592-V08H82)

Furthermore, the luminescence activities between a spike protein of a SARS coronavirus (SARS-CoV) that was a SARS-related coronavirus, and spike proteins of a MERS coronavirus (MERS-CoV) and a human coronavirus (HCoV) that were coronaviruses other than the SARS-related coronavirus, and native Cypridina luciferin were investigated. As monomeric protein samples, the following samples were used. All the samples were manufactured by Sino Biological, Inc., and were monomers. Moreover, the samples were provided in a freeze-dried state.
SARS-CoV Spike S1 (Product number: 40150-V05H1)
MERS-CoV Spike S1 (Product number: HPLC-40069-V08H)
HCoV-HKU1 Spike S1 (Product number: 40021-V08H)
HCoV-NL63 Spike S1 (Product number: 40600-V08H)
HCoV-229E Spike S1 (Product number: 40601-V08H)
HCoV-OC43 Spike S1 (Product number: 40607-V08H1)

The freeze-dried proteins were dissolved in 10 mM PBS (pH: 7.4) to obtain protein solutions.

A solvent (buffer) alone was also measured as a control, but not the protein samples.

A measurement condition was as described below.

### (Measurement Condition)

μL of each of the protein solutions (proteins shown in the drawings: 720 μM) and 45 μL of a luciferin-buffer solution (20 M native Cypridina luciferin, PBS×1) were mixed in a 96-well plate (½ Area OpticalPlate-96, manufactured by PerkinElmer, Inc.), and a signal was immediately measured for 1 minute using a luminescence plate reader (GloMax Explorer Multimode Microplate Reader manufactured by Promega Corporation) to determine the luminescence intensity (Luminescence) ([RLU/min]) (n=4).

Note that the final concentration of the protein in the reaction solution was 72 μg/mL.

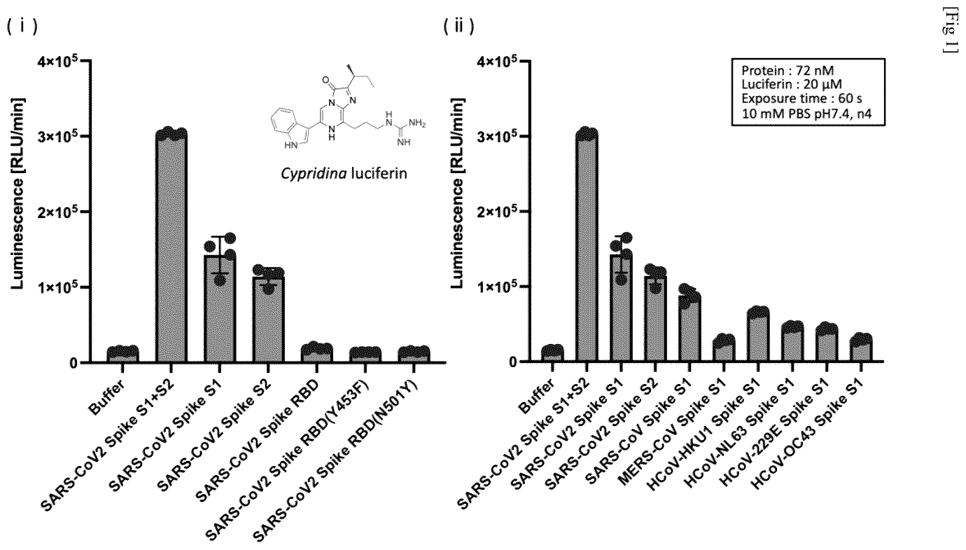

The results are shown in FIG. 1(i) and FIG. 1(ii).

Furthermore, as another protein sample, human serum-derived IgA (IgA; 306-511123, manufactured by FUJIFILM Wako Pure Chemical Corporation) was used, and compared with a SARS coronavirus spike protein (Monomer spike; SARS-CoV-2 Spike S1+S2 (Product number: 40589-V08H04)).

The freeze-dried protein was dissolved in 10 mM PBS (pH: 7.4) to obtain a protein solution.

A solvent (buffer) alone was also measured as a control, but not the protein sample.

A measurement condition was as described below.

### (Measurement Condition)

μL of the protein solution (a protein shown in the drawings: 100 μg/mL) and 45 L of a luciferin-buffer solution (20 μM native Cypridina luciferin, PBS×1) were mixed in a 96-well plate (½ Area OpticalPlate-96, manufactured by PerkinElmer, Inc.), and a signal was immediately measured for 1 minute using a luminescence plate reader (GloMax Explorer Multimode Microplate Reader manufactured by Promega Corporation) (n=4).

Note that the final concentration of the protein in the reaction solution was 10 μg/mL.

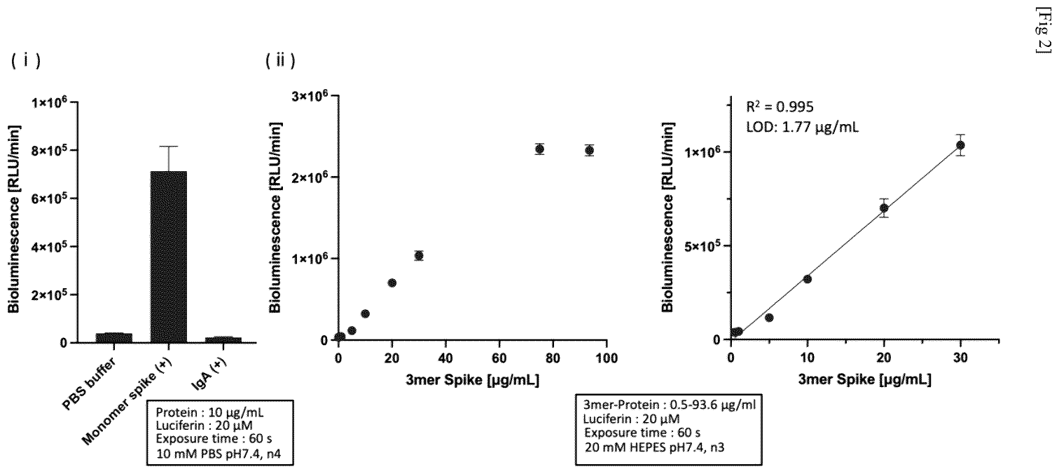

The results are shown in FIG. 2(i).

Consequently, it was found that native Cypridina luciferin caused a luminescent reaction at S1 and S2, whereas native Cypridina luciferin did not cause a reaction at the PBD domain at all (FIG. 1(i)).

In addition, the luminescence activities of different kinds of coronaviruses were examined. As a result, native Cypridina luciferin reacted with spike proteins of SARS-CoV-2 and SARS-CoV, but did not react with the protein of MERS-CoV (FIG. 1(ii)). As been obvious from the results, native Cypridina luciferin can specifically detect the SARS-related coronavirus among the coronaviruses.

Furthermore, it was found that native Cypridina luciferin did not cause a luminescent reaction with the immunoglobulin protein IgA that was present in a large amount in the biological sample such as saliva (FIG. 2(i)).

### 4. Example 3

A correlation between the concentration of a virus spike protein and the luminescence intensity of native Cypridina luciferin was verified.

Measurement was performed in the same manner as in Example 1 except that a trimeric spike protein derived from a SARS-CoV-2 virus (Trimeric SARS-CoV-2 Spike Protein, Full-length, BSV-COV-PR-34, manufactured by M&S TechnoSystems Inc.) was used as a protein solution and the concentration was changed.

The results are shown in FIG. 2(ii).

It was found that the luminescence intensity of native Cypridina luciferin depends on the concentration of the virus spike protein.

### 5. Example 4

By using various types of proteins as protein samples, it was verified that a luminescent reaction specifically occurs between native Cypridina luciferin and a SARS coronavirus spike protein.

As the protein samples, the following samples were used.
Trimeric spike protein derived from a SARS-CoV-2 virus (3-mer; Trimeric SARS-CoV-2 Spike Protein, Full-length, Product number: BSV-COV-PR-34, manufactured by M&S TechnoSystems Inc.)
α-amylase (α-amylase; Product number 017-26371, manufactured by FUJIFILM Wako Pure Chemical Corporation)
Lactoferrin, human (Lactoferrin; Product number: L4040, manufactured by Sigma-Aldrich)
Lysozyme, human, recombinant (plant expression) (Lysozyme; Product number: 181-02063, manufactured by FUJIFILM Wako Pure Chemical Corporation)
Mucin, derived from pig stomach (Mucin; Product number: 137-09162, manufactured by FUJIFILM Wako Pure Chemical Corporation)
Epithelial cell growth factor (EGF), human, recombinant (EGF; Product number: 059-07873, manufactured by FUJIFILM Wako Pure Chemical Corporation)
Human serum-derived IgA (IgA; Product number: 306-51123, manufactured by FUJIFILM Wako Pure Chemical Corporation)

### (Measurement Condition)

5 μL of each of the protein solutions (100 μg/mL) and 45 L of a luciferin-buffer solution (20 M native Cypridina luciferin, PBS×1) were mixed in a 96-well plate (½ Area OpticalPlate-96, manufactured by PerkinElmer, Inc.), and a signal was immediately measured for 1 minute using a luminescence plate reader (GloMax Explorer Multimode Microplate Reader manufactured by Promega Corporation) (n=4).

The results are shown in FIG. 4.

Native Cypridina luciferin did not cause a luminescent reaction with the various proteins.

1. A compound represented by the following general formula (1), or a salt or a solvate thereof: 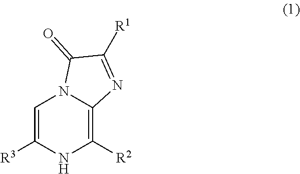
wherein R¹ is a methyl group or any one group represented by the following formula (i-1) or (i-2): 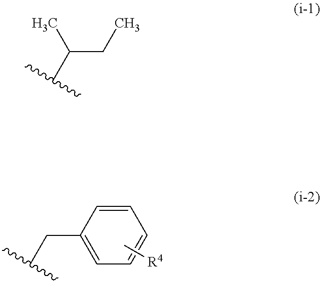
wherein R⁴ is a hydrogen atom, a hydroxy group, a fluorine atom, a methoxy group, or a trifluoromethyl group;
R² is a hydrogen atom or any one group represented by the following formula (ii): 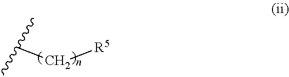
wherein n is an integer of 1 to 5, and R⁵ is any one group represented by the following formula (v-1), (v-2), or (v-3): 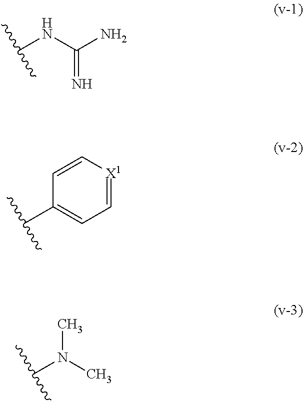
wherein X¹ is a nitrogen atom or a CH group; and
R³ is any one group represented by the following formula (iii-1) or (iii-2): 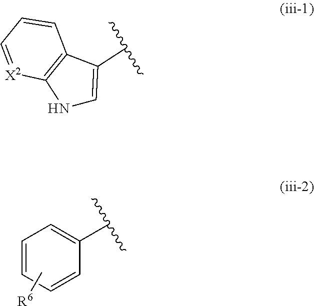
wherein X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5): 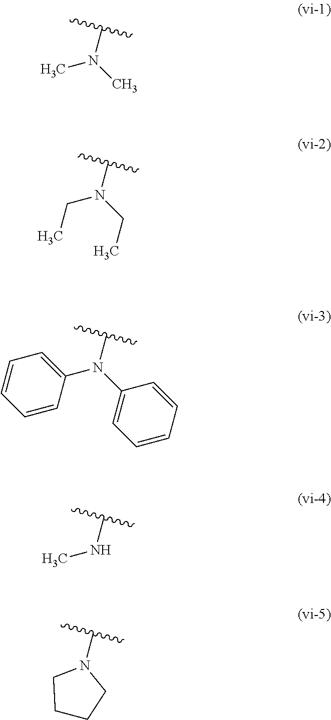
the compound satisfying the following condition (A-i), (A-ii), or (A-iii):
(A-i) the R¹ is a group represented by the following formula (i-2): 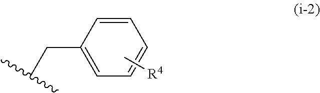
wherein R⁴ is the same as described above, and the R² is a group represented by the following formula (ii): 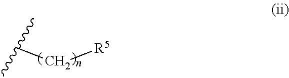
wherein n and R⁵ are the same as described above;
(A-i) the R¹ is a group represented by the following formula (i-1): 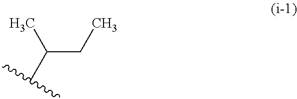 [SMILES: *C(C)CC]
provided that native Cypridina luciferin is excluded; and
(A-iii) the R¹ is a methyl group and the R² is a group represented by the following formula (ii): 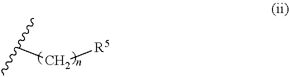
wherein n and R⁵ are the same as described above.

2. The compound or the salt or solvate thereof according to claim 1, wherein the compound represented by the general formula (1) is a compound represented by any of the following formulae.

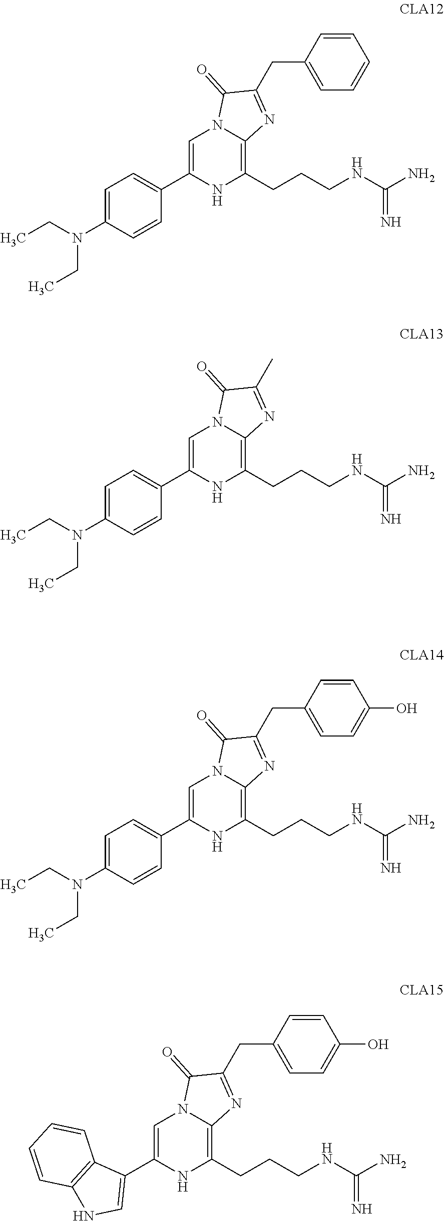

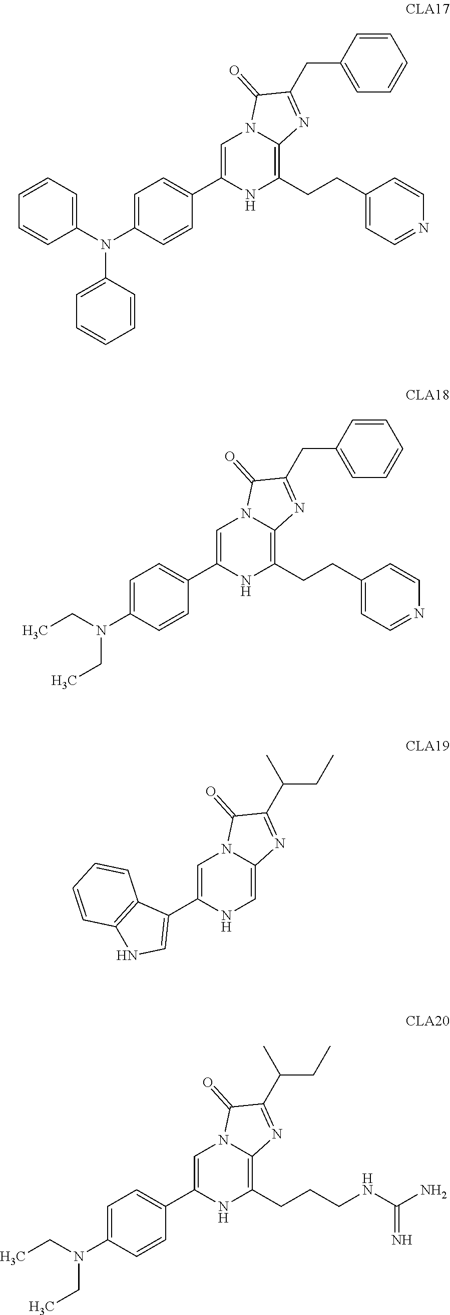

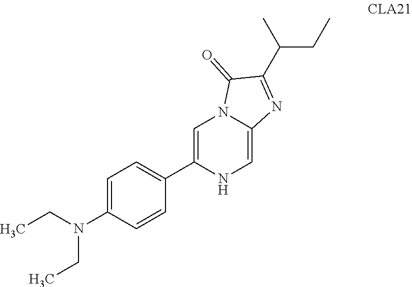

3. A method for detecting a SARS-related coronavirus comprising: a step of bringing a compound represented by the following general formula (2), or a salt or solvate thereof into contact with a biological sample collected from a subject: 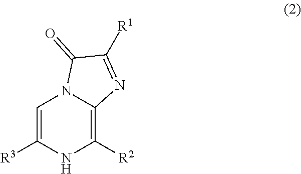
wherein R¹ is a methyl group or any one group represented by the following formula (i-1) or (i-2): 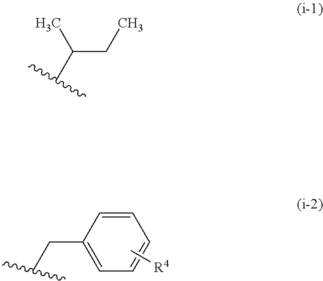
wherein R⁴ is a hydrogen atom, a hydroxy group, a fluorine atom, a methoxy group, or a trifluoromethyl group;
R² is a hydrogen atom or any one group represented by the following formula (ii): 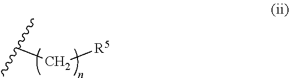
wherein n is an integer of 1 to 5, and R⁵ is any one group represented by the following formula (v-1), (v-2), or (v-3): 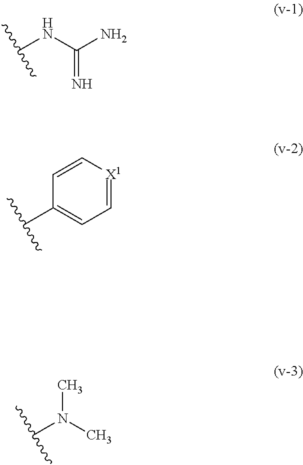
wherein X¹ is a nitrogen atom or a CH group; and
R³ is any one group represented by the following formula (iii-1) or (iii-2): 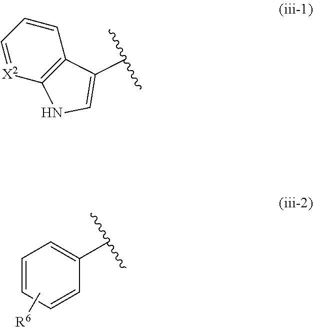
wherein X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5):

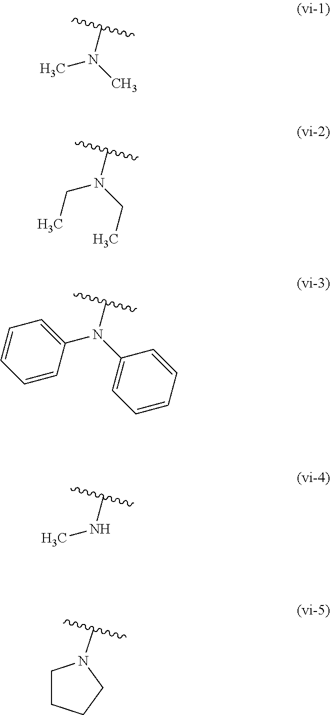

4. The method for detecting a SARS-related coronavirus according to claim 3, wherein the compound represented by the general formula (2) is a compound represented by any of the following formulae.

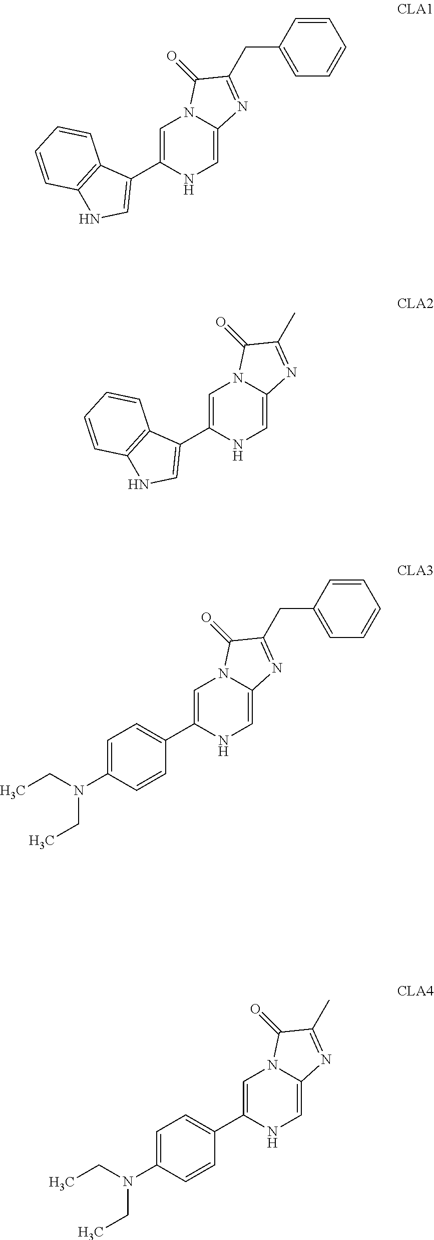

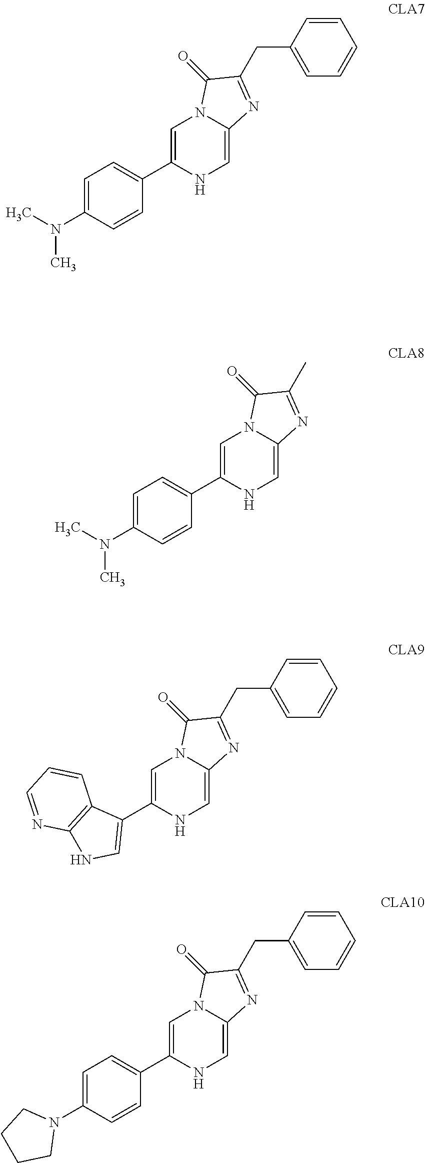

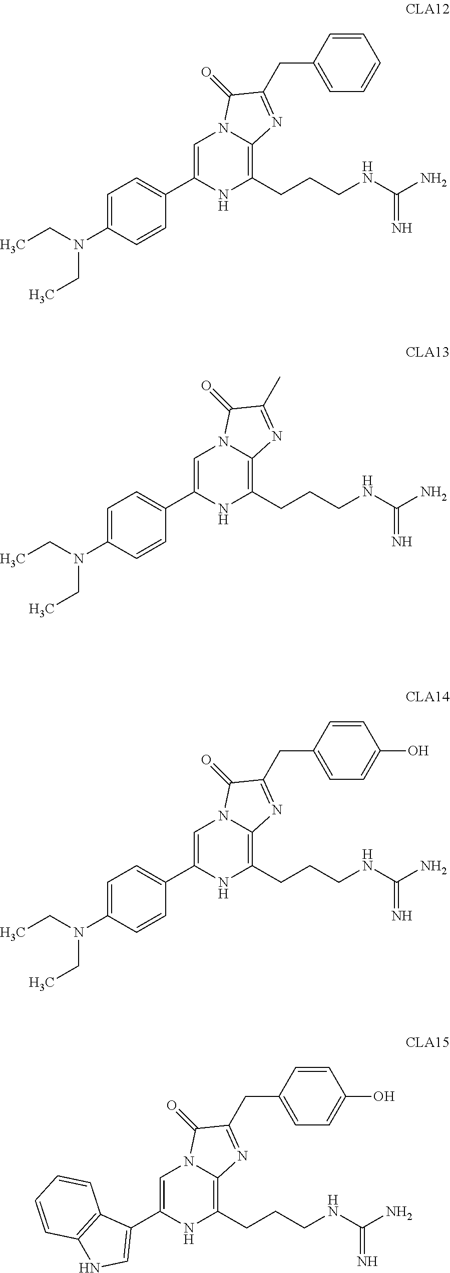

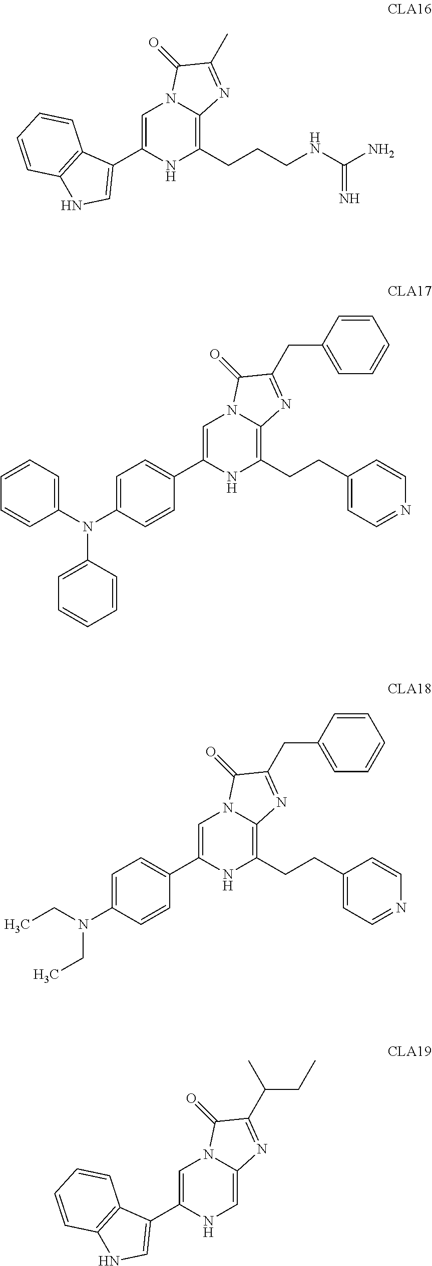

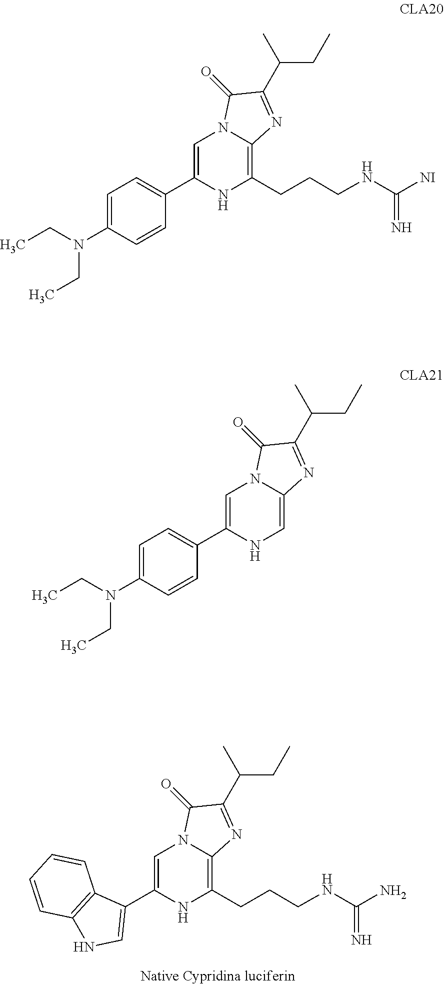

5. The method for detecting a SARS-related coronavirus according to claim 3, wherein the compound represented by the general formula (2) satisfies at least two of the following conditions (B-i), (B-ii), and (B-iii):
(B-i) the R¹ is a group represented by the following formula (i-1): 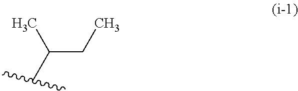 [SMILES: *C(C)CC]
(B-ii) the R² is a group represented by the following formula (ii): 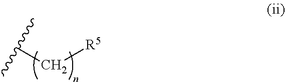
wherein n and R⁵ are the same as described above; and
(B-iii) the R³ is a group represented by the following formula (iii-2-2).

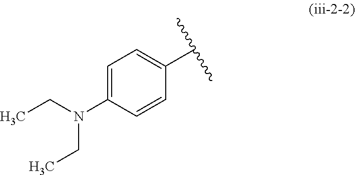 [SMILES: *c1ccc(N(CC)CC)cc1]

6. The method for detecting a SARS-related coronavirus according to claim 3, further comprising: a step of measuring luminescence after the contact step.

7. An agent for detecting a SARS-related coronavirus comprising a compound represented by the following general formula (2), or a salt or solvate thereof: 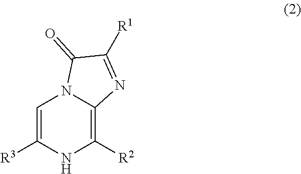
wherein R¹ is a methyl group or any one of groups represented by the following formulae: 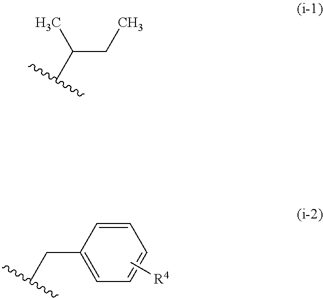
wherein R⁴ is a hydrogen atom, a hydroxy group, a fluorine atom, a methoxy group, or a trifluoromethyl group;
R² is a hydrogen atom or any one group represented by the following formula: 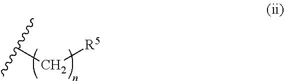
wherein n is an integer of 1 to 5, and R⁵ is any one of groups represented by the following formulae: 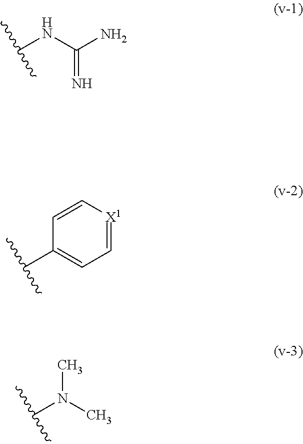
wherein X¹ is a nitrogen atom or a CH group; and R³ is any one of groups represented by the following formulae: 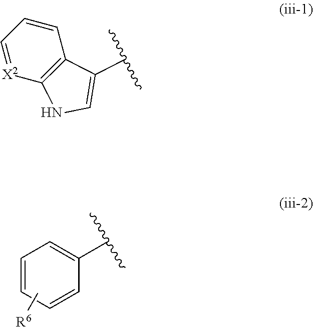
wherein X² is a nitrogen atom or a CH group, and R⁶ is a hydroxy group, a methoxy group, a methyl group, a trifluoromethyl group, or any one group represented by the following formula (vi-1), (vi-2), (vi-3), (vi-4), or (vi-5): [chemical structure]
